(19) United States
(12) Patent Application Publication
(10) Pub. No.: US 2025/0326798 A1
(43) Pub. Date: Oct. 23, 2025

(54) Synthetic Process for Production of Modified GCC Receptor Agonists

(71) Applicant: Ironwood Pharmaceuticals, Inc., Boston, MA (US)

(72) Inventors: Christopher LEITHEISER, Arlington, MA (US); Thomas STORZ, Boston, MA (US); Lin CHEN, Boston, MA (US)

(21) Appl. No.: 18/710,964

(22) PCT Filed: Nov. 22, 2022
(86) PCT No.: PCT/US2022/080295

§ 371 Date: May 16, 2024

Related U.S. Application Data
(60) Provisional application No. 63/282,842, filed on Nov. 24, 2021.
(60) Provisional application No. 63/323,552, filed on Mar. 25, 2022.

(51) Int. Cl. C07K 7/64; A61K 38/00; C07K 1/06; C07K 1/10

(52) U.S. Cl. CPC C07K 7/64; C07K 1/061; C07K 1/10; A61K 38/00

(57) ABSTRACT

The present invention relates to methods of producing a synthetic peptide or pharmaceutically acceptable salts thereof of SEQ ID NO: 1.

### CROSS REFERENCE TO RELATED APPLICATIONS

The present application is a national phase application of PCT/US2022/080295, filed Nov. 22, 2022, which claims priority to and the benefit of U.S. Provisional Application No. 63/282,842, filed Nov. 24, 2021, and to U.S. Provisional Application No. 63/323,552, filed Mar. 25, 2022, the contents of which are herein incorporated by reference in their entireties.

### FIELD OF THE INVENTION

The present invention relates to methods of producing a synthetic peptide or pharmaceutically acceptable salts thereof of SEQ ID NO: 1.

### SEQUENCE LISTING

This application incorporates by reference in its entirety the Sequence Listing entitled “223355-519432.xml” (7.73 kilobytes) which was created on Nov. 21, 2022 at 9:49 AM, and filed electronically herewith.

### BACKGROUND OF THE INVENTION

Interstitial cystitis/bladder pain syndrome (IC/BPS) is a chronic condition involving bladder pain usually accompanied by urinary urgency, increased frequency, and/or nocturia. IC/BPS is often misdiagnosed as a urinary tract infection and antibiotics are generally ineffective. It is estimated that 3-7% of women and 3-4% of men meet the definition of IC/BPS. There may be several contributing factors for the cause of IC/BPS, and it is unknown if IC/BPS is a primary disorder or the secondary result of another disorder [Hanno et al. 2015, 193; 1545-1553]. There are no diagnostic tests for IC/BPS and diagnosis is generally based on urinary symptoms of urgency and frequency accompanied by pain related to the bladder. Diagnosis is generally reserved until other diseases that could cause these symptoms are ruled out.

There are few approved therapies available for IC/BPS. Patients often begin treatment with non-pharmacological treatments (general relaxation, stress management, behavior modification, and physical therapy techniques). Due to the marginally effective therapies available for IC/BPS, many patients utilize off-label therapies including intravesical instillations (i.e., mixtures of medications delivered directly to the bladder through a catheter) to relieve their symptoms. A need exists for more effective, well tolerated treatments for IC/BPS.

A 13-amino-acid, guanylate cyclase C (GC-C) agonist synthetic peptide is being developed for the treatment of bladder pain associated with IC/BPS and, potentially, other visceral pain conditions in the abdominal region. To further the development of this peptide, a need exists for an efficient synthesis and purification process SUMMARY OF THE INVENTION

The present inventions relates to a method of producing a synthetic peptide, or a pharmaceutically acceptable salt thereof. The method having the steps of (i) chemically synthesizing a linear peptide having its C-terminus bound to a solid phase support using a plurality of amino acids and at least one polyamino acid synthon, the linear peptide having protecting groups in one or more amino acids and/or the polyamino acid synthon; wherein at least one amine group of the polyamino acid synthon has a different protecting group from the N-terminus of the linear peptide; (ii) cleaving the linear peptide from the solid phase support to generate a protected peptide; (iii) coupling an amino acid to the C-terminus of the protected peptide; (iv) removing one amine protecting group and one carboxylic acid protecting group of the protected peptide to form a partially unprotected peptide having an unprotected amine and an unprotected carboxylic acid group; (v) coupling the unprotected amine and the unprotected carboxylic acid group to form a cyclized peptide; (vi) globally deprotecting the cyclized peptide to obtain a globally deprotected peptide; (vii) folding the globally deprotected peptide to form one or more additional crosslinks to obtain the synthetic peptide; (viii) optionally, modifying the N-terminus of the synthetic peptide with one or more chemical moieties; and (ix) purifying the synthetic peptide. The synthetic peptide produced by the methods described herein comprises the amino acid sequence: Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1). The synthetic peptide contains a covalent bond between the following amino acid residues of the synthetic peptide: Cys₁ and Cys₆, Cth₂ and Cys₁₀, and Cys₅ and Cys₁₃.

In some embodiments, the method comprises the optional step of (viii) modifying the N-terminus of the synthetic peptide with one or more chemical moieties.

Also disclosed herein is a compound, or pharmaceutically acceptable salt thereof, represented by the following structural formula:

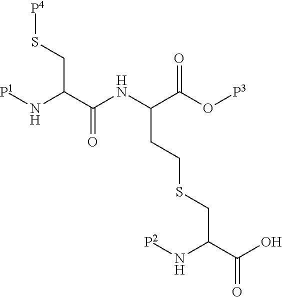

where P¹ and P² are hydrogen or an amine protecting group, provided that when both P¹ and P² are amine protecting groups, they are not the same amine protecting groups; P³ is hydrogen or a carboxylic acid protecting group; and P⁴ is hydrogen or a thiol protecting group.

### BRIEF DESCRIPTION OF THE FIGURES

FIG. 1 shows an exemplary flow diagram for the manufacture of the synthetic peptide of SEQ ID NO: 1.

### DETAILED DESCRIPTION OF THE INVENTION

A method of producing a synthetic peptide, or a pharmaceutically acceptable salt thereof is described herein. The method described herein comprises:
(i) chemically synthesizing a linear peptide having its C-terminus bound to a solid phase support using a plurality of amino acids and at least one polyamino acid synthon, the linear peptide having protecting groups in one or more amino acids and/or the polyamino acid synthon;
wherein at least one amine group of the polyamino acid synthon has a different protecting group from the N-terminus of the linear peptide;
(ii) cleaving the linear peptide from the solid phase support to generate a protected peptide;
(iii) coupling an amino acid to the C-terminus of the protected peptide, wherein the amino acid has an unprotected amine group, a protected carboxylic acid group, and an optionally protected amino acid side-chain;
(iv) removing one amine protecting group and one carboxylic acid protecting group of the protected peptide to form a partially unprotected peptide having an unprotected amine and an unprotected carboxylic acid group;
(v) coupling the unprotected amine and the unprotected carboxylic acid group to form a cyclized peptide;
(vi) globally deprotecting the cyclized peptide to obtain a globally deprotected peptide;
(vii) folding the globally deprotected peptide to form one or more additional crosslinks to obtain the synthetic peptide;
(viii) optionally, modifying the N-terminus of the synthetic peptide with one or more chemical moieties; and
(ix) purifying the synthetic peptide;
wherein the synthetic peptide comprises the amino acid sequence:
   Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); and
where the synthetic peptide contains a covalent bond between the following amino acid residues of the synthetic peptide: a) Cys₁ and Cys₆, b) Cth₂ and Cys₁₀, and c) Cys₅ and Cys₁₃.

### Definitions

As used herein, “Cth” represents cystathionine, which has two α-amino carboxyl groups, designated “1” and “2” in Scheme 1, which can form peptide bonds.

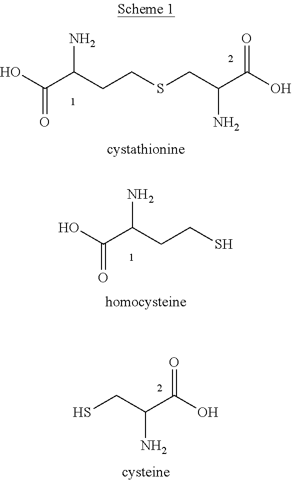 [SMILES: NC(CCSCC(N)C(=O)O)C(=O)O ; NC(CCS)C(=O)O ; NC(CS)C(=O)O]

However, to facilitate the use of the 3-letter amino acid code in describing a peptide sequence, when a cyclic peptide sequence is created by forming a peptide bond with each of the α-amino carboxyl group (designated “1” and “2”) at non-consecutive positions in the peptide sequence, thus creating a cyclic thioether bridge, the peptide bond formed by the α-amino carboxyl group at position 1 is designated “Cth,” whereas the peptide bond formed by the α-amino carboxyl group at position 2 is designated “Cys.” See the section entitled “Synthetic Peptide” for further details.

As used herein, “Hcy” represents homocysteine as shown in Scheme 1. As can be seen from Scheme 1, cystathionine can be viewed as a combination of homocysteine and cysteine, where their side chains share a sulfur atom. Therefore, an alternative method of designating a cyclic peptide sequence, which is created by forming a peptide bond with each of the α-amino carboxyl group of cystathionine at non-consecutive positions in the peptide sequence, is by designating the peptide linkage formed by the α-amino carboxyl group at position 1 “Hcy” and the peptide linkage formed by the α-amino carboxyl group at position 2 “Cys.”

As used herein, unless other indicated, “pharmaceutically acceptable” means biologically or pharmacologically compatible for in vivo use in animals or humans, and preferably means, approved by a regulatory agency of the Federal or a state government or listed in the U.S. Pharmacopeia or other generally recognized pharmacopeia for use in animals, and more particularly in humans.

As used here, unless otherwise indicated, the terms “about” and “approximately” mean within an acceptable error range for the particular value as determined by one of ordinary skill in the art, which will depend, in part, on how the value is measured or determined, i.e., the limitations of the measurement system. For example, “about” can mean within 1 or more than 1 standard deviation, per practice in the art. Alternatively, “about” with respect to the compositions can mean plus or minus a range of up to 20%, preferably up to 10%. Alternatively, particularly with respect to biological systems or processes, the term can mean within an order of magnitude, preferably within 5-fold, and more preferably within 2-fold, of a value. Particular values are described in the application and claims, unless otherwise stated the term “about” means within an acceptable error range for the particular value.

### Synthetic Peptide

In some embodiments, the synthetic peptide produced by the methods of the present disclosure can be linearly represented as Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1).

The synthetic peptide of SEQ ID NO: 1 contains four cysteine residues that form two disulfide bonds, and a cystathione (Cth) unit (combining homocysteine and cysteine, which share the side chain sulfur atom) providing an internal sulfide (or thioether) bond, with the defined connectivity (Cys₁-Cys₆, Cys₅-Cys₁₃, Cth₂-Cys₁₀).

For purposes of the present description, the two parts of the linear sequence are designated as Cth₂ and Cys₁₀, where the thioether bond connects the sulfur a homocysteine (Hcy) side chain and a carbon of a des-SH cysteine side chain: this double amino-acid corresponds to a cystathionine (Cth) residue, but the proposed designation facilitates the description when using the 3-letter code designation of the residues, where the peptide linkage formed by the α-amino carboxyl group of position 1 is designated “Cth” and the peptide linkage formed by α-amino carboxyl group of position 2 is designated “Cys,” see Scheme 1 above.

Alternatively, and for purposes of the present description, the two parts of the building blocks may be designated, respectively, as [Hcy] and [Cys] where the sulfur of the homocysteine (Hcy) side chain is shared with a side chain of a cysteine (Cys) to form a thioether bridge: this double amino-acid corresponds to a cystathionine (Cth) residue, but the proposed designation facilitates the description when using the 3-letter code designation of the residues. Using this alternative nomenclature SEQ ID NO 1 can be represented as follows: Cys₁ Hcy₂ Glu₃ Leu₄ Cys₅ Cys₆ Asn₇ Val₈ Ala₉ Cys₁₀ Tyr₁₁ Gly₁₂ Cys₁₃ (SEQ ID NO: 1).

In some embodiments, the designation of Cth₂-Cys₁₀, or any variation thereof, is meant to describe the linkage between the side chains of two non-consecutive amino acids in SEQ ID NO 1 which forms a thioether bridge as shown below:

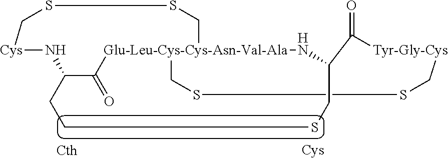

In some embodiments, the designation of Cth₂-Cys₁₀, or any variation thereof, describes a cystathionine which forms a peptide bond at positions 2 and 10 of the synthetic peptide and forms a thioether bridge.

In some embodiments, the synthetic peptide of SEQ ID NO: 1 can be represented by the formula:

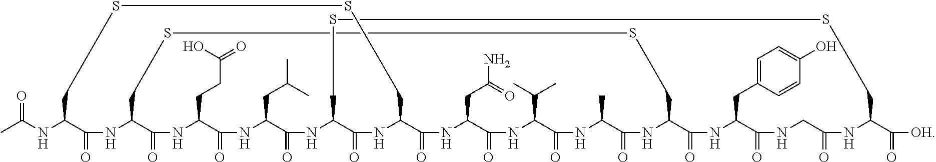 [SMILES: CC(=O)N[C@H]1CSSC[C@@H]2NC(=O)[C@@H]3CSSC[C@@H](C(=O)O)NC(=O)CNC(=O)[C@H](Cc4ccc(O)cc4)NC(=O)[C@H](CSSC[C@H](NC1=O)C(=O)N[C@@H](CCC(=O)O)C(=O)N[C@@H](CC(C)C)C(=O)N3)NC(=O)[C@H](C)NC(=O)[C@H](C(C)C)NC(=O)[C@H](CC(N)=O)NC2=O]

### Method of Producing a Synthetic Peptide

The method described herein begins by (i) chemically synthesizing a linear peptide having its C-terminus bound to a solid phase support using a plurality of amino acids and at least one polyamino acid synthon, the linear peptide having protecting groups in one or more amino acids and/or the polyamino acid synthon. In some embodiments, at least one amine group of the polyamino acid synthon has a different protecting group from the N-terminus of the linear peptide.

In some embodiments, the solid phase support is selected from the group consisting of Wang resins, Trityl resins, and Rink resins.

In some embodiments, the solid phase support has a loading of about 0.10 mmol/g, about 0.20 mmol/g, about 0.30 mmol/g, about 0.40 mmol/g, about 0.50 mmol/g, about 0.60 mmol/g, about 0.70 mmol/g, about 0.80 mmol/g, about 0.90 mmol/g, or about 1.00 mmol/g. In some embodiments, the solid phase has a loading of about 0.70 mmol/g. In some embodiments, the solid phase has a loading of about 0.90 mmol/g.

In some embodiments, the polyamino acid synthon is a compound represented by the formula:

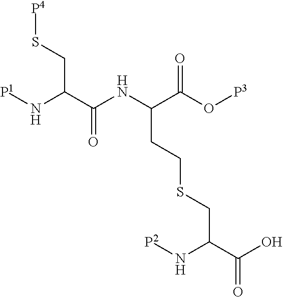

where P¹ and P² are hydrogen or an amine protecting group, provided that when both P¹ and P² are amine protecting groups they are not the same amine protecting group; P³ is hydrogen or a carboxylic acid protecting group; and P⁴ is hydrogen or a thiol protecting group.

In a preferred embodiment, P¹ and P² are different amine protecting groups; P³ is a carboxylic acid protecting group; and P⁴ is a thiol protecting group.

In some embodiments, the protecting groups are selected from the group consisting of fluorenylmethyloxycarbonyl (Fmoc), tert-butyloxycarbonyl (Boc), carboxybenzyl (Cbz), trityl, methyl, ethyl, tert-Butyl, allyl (All), 2,4-dimethoxybenzyl (Dmb), 9-fluorenylmethyl (Fm), benzyl (Bn), tert-butyldimethylsilyl, allyloxycarbonyl (alloc), tert-butyloxycarbonyl, acetamidomethyl (Acm), 3-nitro-2-pyridine sulfenyl (NPYS), and 2-pyridine-sulfenyl (Pyr).

In some embodiments, the amine protecting groups, P¹ and P², are each selected from the group consisting of fluorenylmethyloxycarbonyl (Fmoc), tert-butyloxycarbonyl (Boc), and carboxybenzyl (Cbz). In some embodiments, P¹ or P² is a tert-butyloxycabonyl (Boc) protecting group. In some embodiments, P¹ or P² is a 9-fluorenylmethoxycarbonyl (Fmoc) protecting group. In some embodiments, P¹ is a tert-butyloxycabonyl (Boc) protecting group and P² is a 9-fluorenylmethoxycarbonyl (Fmoc) protecting group.

In some embodiments, the carboxylic acid protecting group, P³, is selected from the group consisting of methyl, ethyl, tert-Butyl, allyl (All), 2,4-dimethoxybenzyl (Dmb), 9-fluorenylmethyl (Fm), benzyl (Bn). In some embodiments, P³ is an allyl (All) protecting group.

In some embodiments, P⁴ is a trityl protecting group.

In some embodiments, the subunits of the polyamino acid synthon have a D-configuration, e.g., the synthon is a D-Enantiomer. In some embodiments, the polyamino acid synthon with subunits of a D-configuration can be represented by the following formula:

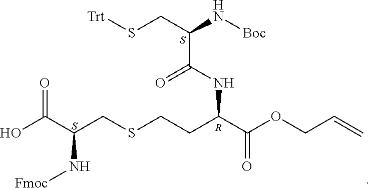

In some embodiments, the subunits of the polyamino acid synthon have an L-configuration, e.g., the synthon is an L-Enantiomer. In some embodiments, the polyamino acid synthon with subunits of a L-configuration can be represented by the following formula:

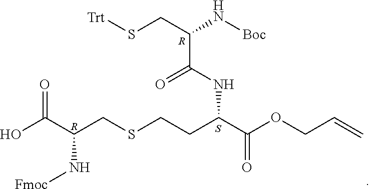

In some embodiments, the subunits of the polyamino acid synthon have both a D-configuration and an L-configuration.

In some embodiments, the amino acid side chains of the linear peptide have a protecting group. In some embodiments, the amino acid side chain protecting groups are selected from the group consisting of tert-Butyl (tBu), trityl (Trt), allyl (All), cyclohexyl, 2-phenylisopropyl, acetamidomethyl (Acm), benzyl (Bzl), 4-methylbenzyl (4-MeBzl), 4-methoxybenzyl (4-MeOBzl), 9-fluorenylmethyl (Fm), tert-butylthio (t-Buthio), 4-methoxytrityl (Mmt), xanthyl (Xan), 2,6-Dichlorobenzyl (2,6-Cl₂Bzl), and 2-bromobenzylcarbonate (2-BrZ). In some embodiments, the amino acid side chain protecting group is tert-Butyl (tBu) or trityl (Trt).

In some embodiments, the amino acid side chains of the linear peptide that have a protecting group on their side chains are Cys₁, Glu₃, Cys₅, Cys₆, Asn₇, Tyr₁₁, and Cys₁₃ of SEQ ID NO: 1.

In some embodiments, the plurality of amino acids and the synthon are coupled by a carbodiimide-mediated reaction or by a reaction mediated by a non-carbodiimide coupling agents, for example, 1-[Bis(dimethylamino)methylene]-1H-1,2,3-triazolo[4,5-b]pyridinium 3-oxide hexafluorophosphate (HATU), (2-(1H-benzotriazol-1-yl)-1,1,3,3-tetramethyluronium hexafluorophosphate (HBTU), 1H-Benzotriazolium 1-[bis(dimethyl-amino)methylene]-5-chloro-hexafluorophosphate (1-),3-oxide (HCTU), O-(Benzotriazol-1-yl)-N,N,N′,N′-tetramethyluronium tetrafluoroborate (TBTU), 1-[(1-(cyano-2-ethoxy-2-oxoethylideneaminooxy)-dimethylamino-morpholinomethylene)]methanaminium hexafluorophosphate (COMU), 1-Cyano-2-ethoxy-2-oxoethylidenaminooxy-tris-pyrrolidino-phosphonium hexafluorophosphate (PyOxim), benzotriazol-1-yloxytripyrrolidinophosphonium hexafluorophosphate (PyBOP), 7-Azabenzotriazol-1-yloxy)tripyrrolidinophosphonium hexafluorophosphate (PyAOP), or propanephosphonic acid anhydride (T3P) to form the linear peptide of step (i).

In some embodiments, at least one amino acid from the plurality of peptides and/or the synthon are coupled by a carbodiimide-mediated reaction to form the linear peptide of step (i). In some embodiments, the carbodiimide is selected from the group consisting of diisopropylcarbodiimide (DIC), dicyclohexylcarbodiimide (DCC), and 1-Ethyl-3-(3-dimethylaminopropyl)carbodiimide (EDC). In some embodiments, the carbodiimide is DIC.

In some embodiments, the carbodiimide-mediated reaction mixture further comprises an amino acid racemization suppressing agent. In some embodiments, the racemization suppressing agent is selected from the group consisting of 2-Hydroxypyridine-N-oxide (HOPO), 1-hydroxybenzotriazole (HOBt), 1-hydroxy-7-azo-benzotriazole (—At), and 2-cyano-2-(hydroxyimino)acetate). In some embodiments, the racemization suppressing agent is 2-cyano-2-(hydroxyimino)acetate).

In some embodiments, the solvent for the carbodiimide-mediated reaction is, but not limited to, N-methylpyrrolidinone (NMP), dichloromethane (DCM), chloroform, or dimethylformamide (DMF). In some embodiments, the solvent for the carbodiimide-mediated reaction is N-methylpyrrolidinone (NMP).

In some embodiments, pyridine is used with the carbodiimide-mediated reaction to prevent premature cleavage of the linear peptide from the solid support.

In some embodiments, at least one amino acid is coupled by a non-carbodiimide coupling agent. In some embodiments, the non-carbodiimide coupling agent is TBTU.

The linear peptide of step (i) may be referred to in this application as a “linear 12-mer” and can be represented by the following formula:

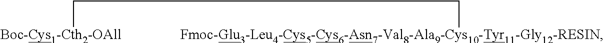

(SEQ ID NO: 2) wherein the cystathionine thioether side chain bridge —CH₂—CH₂—S—CH₂— is represented by “ [symbol] ”. In some embodiments, one or more of the side chains of the underlined amino acids are protected. In some embodiments, the side chains of all the underlined amino acids are protected.

After the linear peptide of step (i) is formed, the linear peptide is (ii) cleaved from the solid phase support to generate a protected peptide in step (ii).

In some embodiments, the linear peptide is cleaved from the resin via treatment with a dilute acidic solution. In some embodiments, the treatment with a dilute acidic solution preserves the side chain protecting groups and the protecting groups of the polyamino acid synthon. In some embodiments, the dilute acidic solution is, for example, dilute trifluoroacetic acid (TFA) or dilute Bromotrimethylsilane (TMSBr). In some embodiments, the dilute acid solution is a TFA solution. In some embodiments, the dilute acid solution is a 1% trifluoroacetic acid (TFA) in dichloromethane (DCM) solution.

Following the cleavage of the linear peptide from the resin to generate a protected peptide, an amino acid which has an unprotected amine group, a protected carboxylic acid group, and an optionally protected amino acid side-chain is coupled to the C-terminus of the protected peptide in step (iii).

In some embodiments, the protected peptide obtained in step (ii) is used as is in step (iii).

In some embodiments, the amino acid coupled to the C-terminus of the protected peptide is a cysteine. In some embodiments, the cysteine is protected. In some embodiments, the amino acid coupled to the C-terminus is S-trityl-L-cysteinyl-O-t-butyl-ester. In some embodiments, the protected peptide obtained in step (ii) may be represented by the following formula:

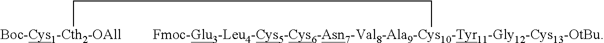

(SEQ ID NO: 3). In some embodiments, one or more of the side chains of the underlined amino acids are protected. In some embodiments, the side chains of all the underlined amino acids are protected.

Following the coupling of the amino acid to the C-terminus in step (iv), one amine protecting group and one carboxylic acid protecting group are removed from the protected peptide formed in step (iii) to form a partially unprotected peptide having an unprotected amine and an unprotected carboxylic acid group.

In some embodiments, the carboxylic acid, which is deprotected in step (iv) derives from the protected carboxylic acid group of the polyamino synthon.

Once the protecting groups are removed, in step (v) the unprotected amine and the unprotected carboxylic acid group are coupled to form a cyclized peptide.

Following the formation of the cyclized peptide in step (v), in step (vi) the cyclized peptide is globally deprotected to obtain a globally deprotected peptide. In some embodiments, the global deprotection step (vi) comprises addition of a cocktail comprising at least ammonium iodide (NH₄I) and thioanisole.

Once the cyclized peptide is globally deprotected, in step (vii) the peptide is folded to form one or more additional crosslinks to obtain the synthetic peptide of Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1).

In some embodiments, the crosslinked synthetic peptide formed in step (vii) contains a covalent bond between the following amino acid residues of the synthetic peptide: Cys₁ and Cys₆, Cth₂ and Cys₁₀, and Cys₅ and Cys₁₃. In some embodiments, the covalent bond between Cys₁ and Cys₆ and Cys₅ and Cys₁₃ is a disulfide bond. In some embodiments, the covalent bond between Cth₂ and Cys₁₀ is a thioether bond.

Following the folding of the globally deprotected peptide, the synthetic peptide of SEQ ID NO: 1 is purified.

In some embodiments, the method includes the optional step (viii) of modifying the N-terminus of the synthetic peptide with one or more chemical moieties. In some embodiments, the N-terminus of the synthetic peptide is modified with an acetyl group. In cases where the N-terminus of the synthetic peptide is modified with one or more chemical moieties, the synthetic peptide is purified twice, once immediately following the folding step (vii) and once after the N-terminal modification.

In some embodiments, the method further comprises precipitating the synthetic peptide from solution. In some embodiments, the precipitation step comprises an acidification step followed by a dilution step with an organic solvent mixture. In some embodiments, the organic solvent mixture comprises at least one of acetonitrile or methyl tert-butyl ether (MTBE).

Also described herein is a method of preparing a synthetic peptide of Formula I:

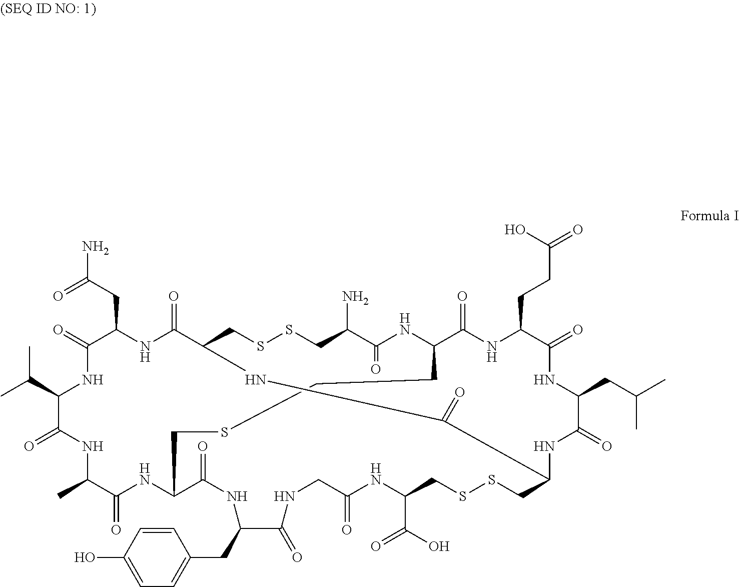 [SMILES: CC(C)C[C@@H]1NC(=O)[C@H](CCC(=O)O)NC(=O)[C@H]2CCSC[C@H]3NC(=O)[C@@H](C)NC(=O)[C@@H](C(C)C)NC(=O)[C@@H](CC(N)=O)NC(=O)[C@@H](CNC(=O)[C@H](CSSC[C@@H](C(=O)O)NC(=O)CNC(=O)[C@@H](Cc4ccc(O)cc4)NC3=O)NC1=O)CSSC[C@@H](N)C(=O)N2]

the method comprising
(i) coupling a C-terminal resin bound Tyr-Gly peptide, wherein the Tyr amino acid residue is protected, to a polyamino acid synthon of Formula II:

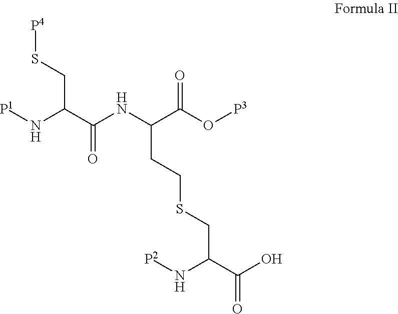

wherein:
P¹ and P² are different amine protecting groups;
P³ is a carboxylic acid protecting group; and
P⁴ is a thiol protecting group,
to form a resin bound peptide of Formula III:

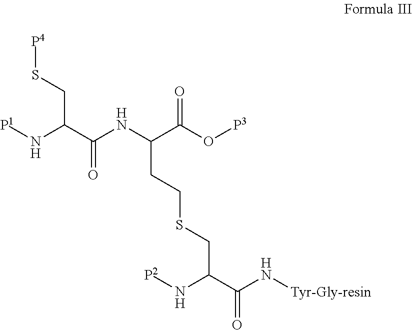

(ii) removing the P² protecting group of Formula III to obtain a resin bound peptide of Formula IV having an unprotected amine group:

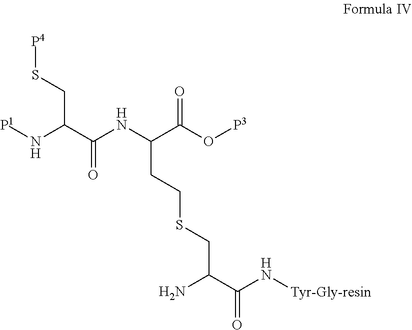

(iii) coupling a P²-alanine to the resin bound peptide of Formula IV via the free amine group of Formula IV to form a resin bound peptide of Formula V:

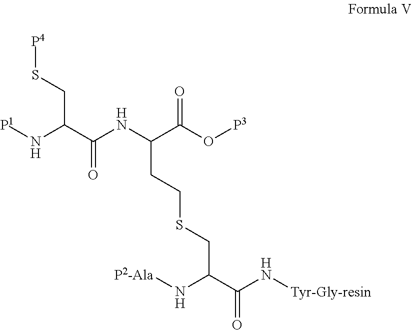

(iv) removing the P² protecting group of Formula V to obtain a free amine group, followed by coupling the free amine group to P²-amino acid, wherein the side chain of the amino acid may be protected;
(v) repeating step (iv) for six more times to form a resin bound peptide of Formula VI:

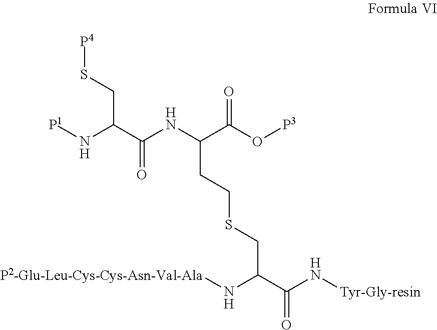

wherein at least one amino acid side chain is protected;
(vi) cleaving the peptide of Formula VI from the resin to form a linear peptide having a C-terminal carboxylic acid group;
(vii) coupling the C-terminal carboxylic acid group of the linear peptide to the amine group of a cysteine,
   wherein the cysteine comprises a carboxylic acid protecting group, and
   wherein the side chain of the cysteine amino acid may be protected, to obtain a protected peptide of Formula VII

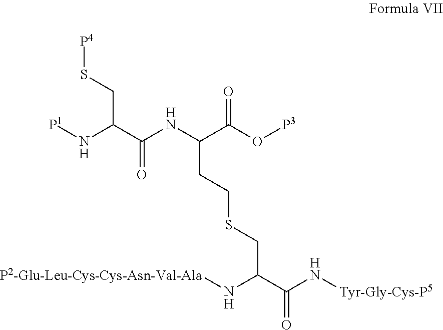

wherein P⁵ is a carboxylic acid protecting group that is different from P³;
(viii) removing the P² protecting group and the P³ protecting group to obtain a free amine group and a free carboxylic acid group;
(ix) coupling the free amine group and the free carboxylic acid group, to obtain a cyclized peptide of Formula VIII:

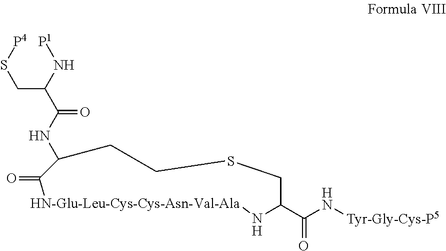

(x) globally deprotecting the cyclized peptide to obtain a globally deprotected peptide; and
(xi) folding the globally deprotected peptide by forming two disulfide bonds to obtain the synthetic peptide of Formula I.

As used herein, P²-amino acid is an amino acid in which the amine group is protected with an amine protecting group which is a different amine protecting group than the amine protecting group of P¹. For example, P²-alanine would have the following structural formula:

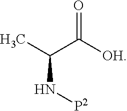

In some embodiments, the method further comprises acetylating a free amine group in Formula I to obtain a synthetic peptide of Formula IX:

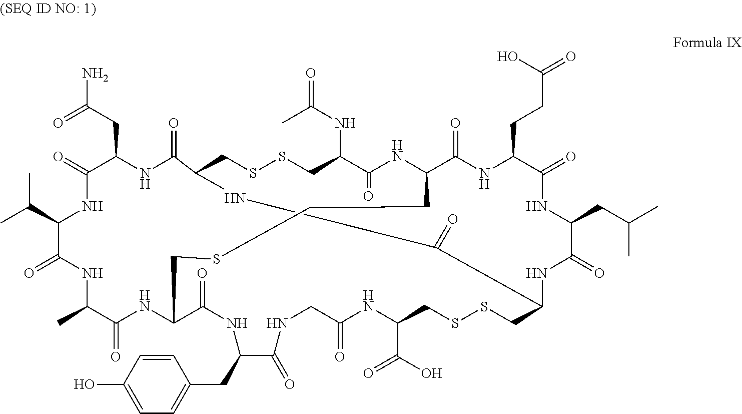 [SMILES: CC(=O)N[C@@H]1CSSC[C@@H]2NC(=O)[C@@H]3CSSC[C@@H](C(=O)O)NC(=O)CNC(=O)[C@@H](Cc4ccc(O)cc4)NC(=O)[C@@H](CSCC[C@@H](NC1=O)C(=O)N[C@@H](CCC(=O)O)C(=O)N[C@@H](CC(C)C)C(=O)N3)NC(=O)[C@@H](C)NC(=O)[C@@H](C(C)C)NC(=O)[C@@H](CC(N)=O)NC2=O]

In some embodiments, P¹ is an acetyl group and steps (i)-(ix) are performed as described above. However, in step (x) during the global deprotection step the acetyl group represented by P¹ is not removed and step (x) the folding step forms a compound represented by Formula IX.

In some embodiments, the Glu, Cys, Cys, Asn, Gly, and Cys residues of Formula VII have side chain protecting groups. In some embodiments, the amino acid side chain protecting groups are selected from the group consisting of tert-Butyl (tBu), trityl (Trt), allyl (All), cyclohexyl, 2-phenylisopropyl, acetamidomethyl (Acm), benzyl (Bzl), 4-methylbenzyl (4-MeBzl), 4-methoxybenzyl (4-MeOBzl), 9-fluorenylmethyl (Fm), tert-butylthio (t-Buthio), 4-methoxytrityl (Mmt), xanthyl (Xan), 2,6-Dichlorobenzyl (2,6-Cl₂Bzl), and 2-bromobenzylcarbonate (2-BrZ). In some embodiments, the amino acid side chain protecting group is tert-Butyl (tBu) or trityl (Trt).

In some embodiments, P¹ and P² are each protecting groups selected from the group consisting of fluorenylmethoxycarbonyl (Fmoc), tert-butyloxycarbonyl (Boc), carboxybenzyl (Cbz), and allyloxycarbonyl (Alloc). In some embodiments, P¹ is a tert-butyloxycarbonyl (Boc) protecting group. In some embodiments, P² is a fluorenylmethoxycarbonyl (Fmoc) protecting group.

In some embodiments, P³ is a protecting group selected from the group consisting of methyl, ethyl, tert-Butyl, allyl (All), trityl, 2,4-dimethoxybenzyl (Dmb), 9-fluorenylmethyl (Fm), and benzyl (Bn). In some embodiments, P³ is an allyl (All) protecting group.

In some embodiments, P⁴ is a protecting group selected from the group consisting of acetamidomethyl (Acm), tert-butyl (t-Bu), 3-nitro-2-pyridine sulfenyl (NPYS), 2-pyridine-sulfenyl (Pyr), and trityl (Trt). In some embodiments, P⁴ is a trityl protecting group. In some embodiments, P⁴ is tert-butyl protecting group.

### Compound

Also disclosed herein is a compound, or pharmaceutically acceptable salt thereof, represented by the following structural formula:

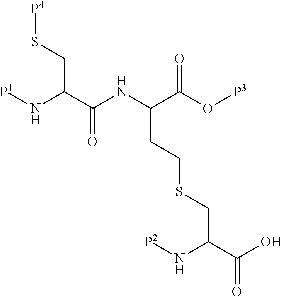

wherein:
   P¹ and P² are hydrogen or an amine protecting group, provided that when both P¹ and P² are amine protecting groups, they are not the same amine protecting groups;
   P³ is hydrogen or a carboxylic acid protecting group; and
   P⁴ is hydrogen or a thiol protecting group.

In some embodiments, at least one of P¹, P², P³, and/or P⁴ is a hydrogen. In some embodiments, P¹ to P⁴ are each hydrogen.

In some embodiments, P¹ to P⁴ are each a protecting group (e.g., P¹ and P² are each amine protecting groups, P³ is a carboxylic acid protecting group, and P⁴ is a thiol protecting group).

In some embodiments, P¹ and P² are each amine protecting groups selected from the group consisting of acetyl, fluorenylmethoxycarbonyl (Fmoc), tert-butyloxycarbonyl (Boc), carboxybenzyl (Cbz), and allyloxycarbonyl (Alloc). In some embodiments, P¹ is an acetyl group. In some embodiments, P¹ is a tert-butyloxycarbonyl (Boc) protecting group. In some embodiments, P² is an Fmoc protecting group.

In some embodiments, P³ is a carboxylic acid protecting group selected from the group consisting of methyl, ethyl, tert-Butyl, allyl (All), trityl, 2,4-dimethoxybenzyl (Dmb), 9-fluorenylmethyl (Fm), and benzyl (Bn). In some embodiments, P³ is an allyl (All) protecting group.

In some embodiments, P⁴ is a thiol protecting group selected from the group consisting of acetamidomethyl (Acm), tert-butyl (t-Bu), 3-nitro-2-pyridine sulfenyl (NPYS), 2-pyridine-sulfenyl (Pyr), and trityl (Trt). In some embodiments, P⁴ is a trityl protecting group.

In some embodiments, the compound, or pharmaceutically acceptable salt thereof, is a compound of Formula A:

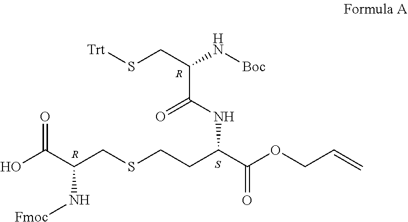

In some embodiments, to arrive at the compound of Formula A, three building blocks are synthesized separately (Parts A-C) and combined (Part D) to synthesize the compound of Formula A, as shown in the schemes below:

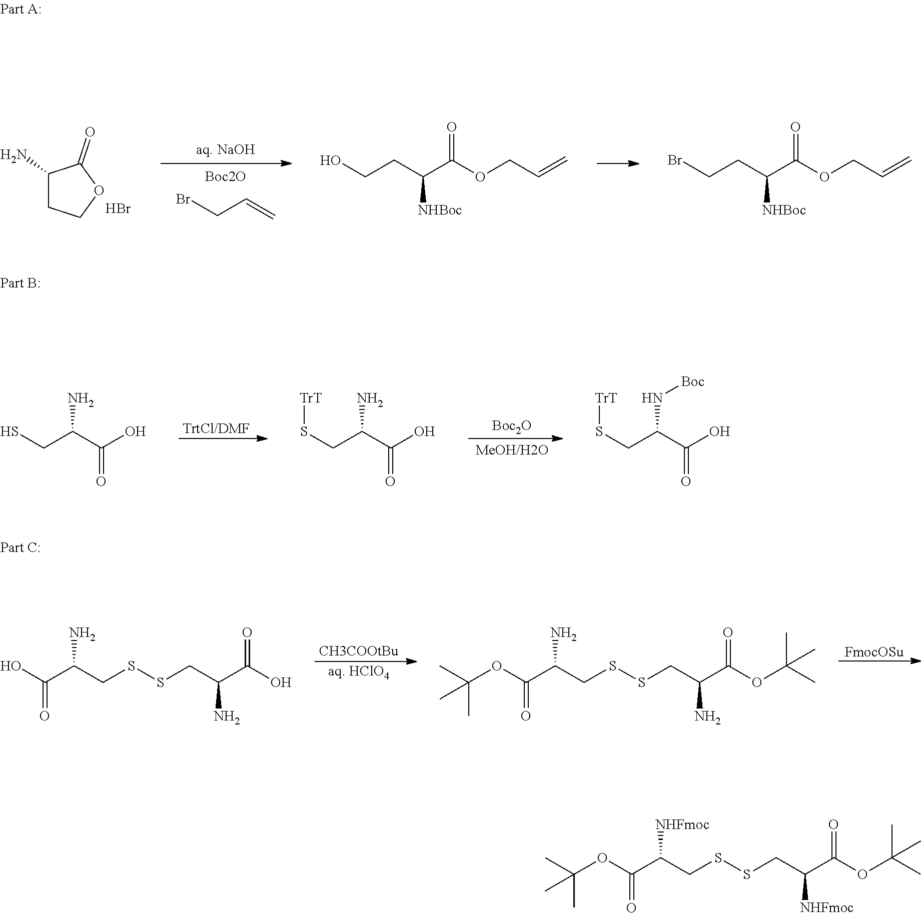

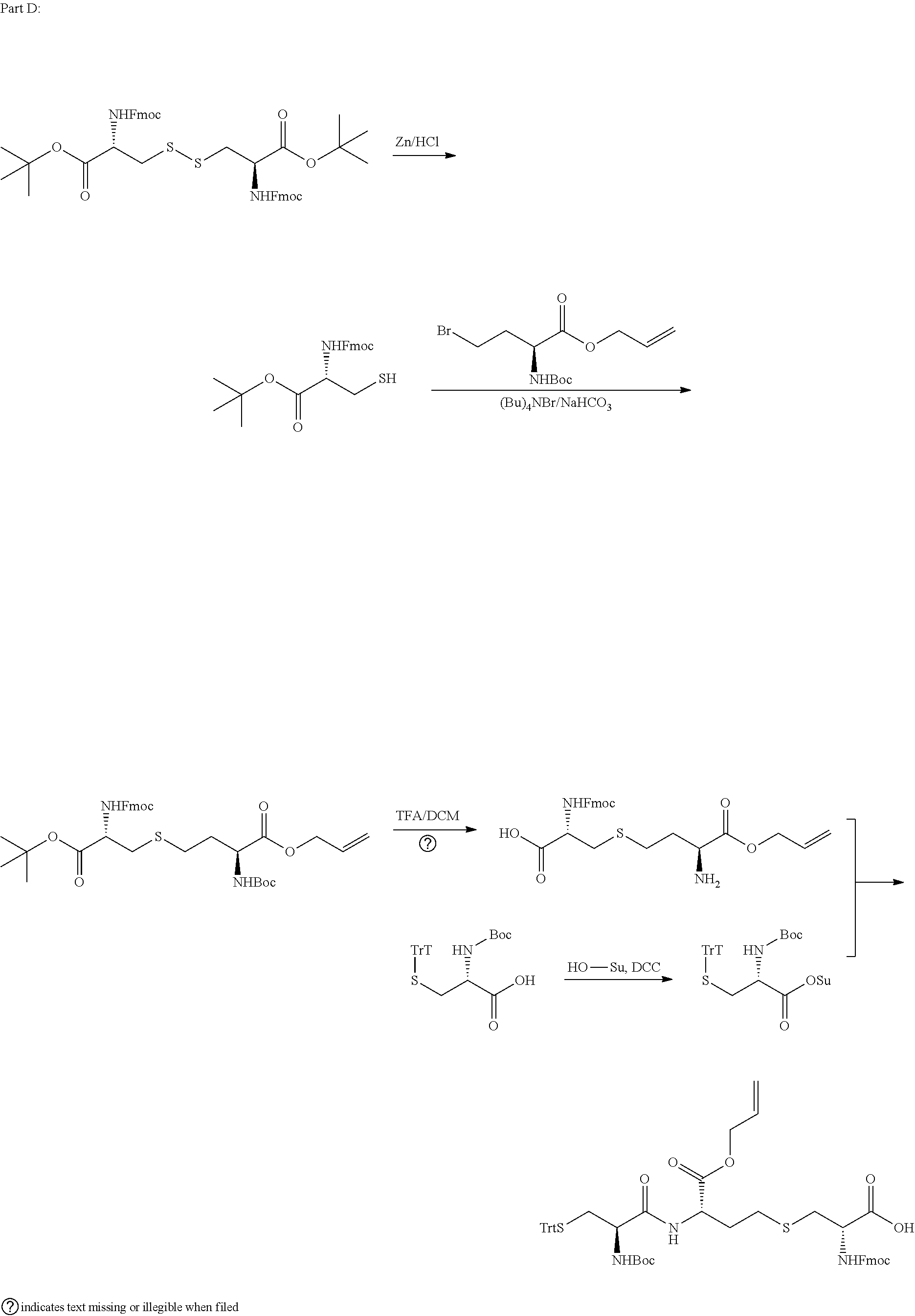

In some embodiments, to arrive at the compound of Formula A, a di-block compound (Alloc-HCys((Fmoc-Ala-OH)3-yl)-OAll) is first synthesized, as shown in the scheme below:

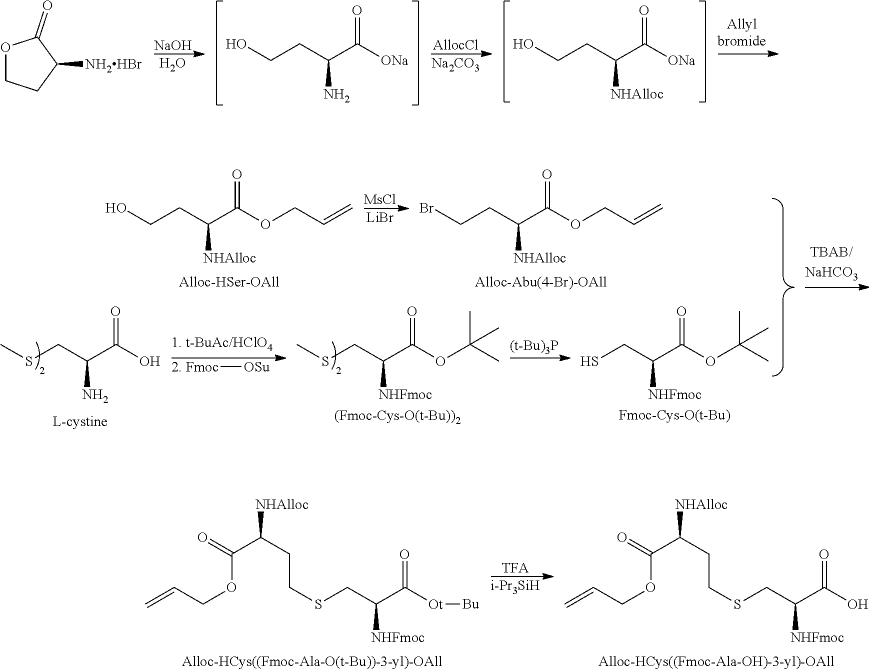

### EXAMPLES

The following examples are merely illustrative of the present invention and should not be construed as limiting the scope of the invention in any way as many variations and equivalents that are encompassed by the present invention will become apparent to those skilled in the art upon reading the present disclosure.

### Reagents and Solvents

The starting materials, reagents, and solvents used in the manufacture of the claimed peptide are listed in Tables 1-3, respectively.

TABLE 1
List of Starting Materials

| Abbreviation | Name and structure |
|---|---|
| Fmoc-L-Ala-OH | Fluorenylmethyloxycarbonyl-L-alanine |
| | 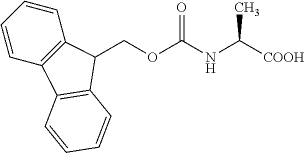 [SMILES: C[C@H](NC(=O)OCC1c2ccccc2-c2ccccc21)C(=O)O] |
| Fmoc-L-Asn(Trt)-OH | Fluorenylmethyloxycarbonyl-N-trityl-L-asparagine |
| | 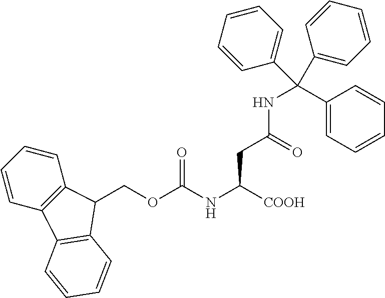 [SMILES: O=C(C[C@H](NC(=O)OCC1c2ccccc2-c2ccccc21)C(=O)O)NC(c1ccccc1)(c1ccccc1)c1ccccc1] |
| Fmoc-L-Cys(Trt)-OH | Fluorenylmethyloxycarbonyl-S-trityl-L-cysteine |
| | 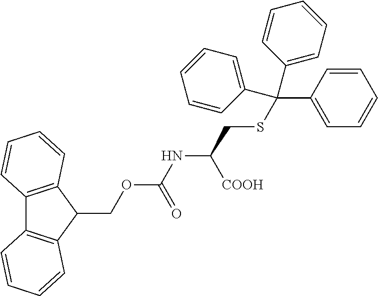 [SMILES: O=C(N[C@@H](CSC(c1ccccc1)(c1ccccc1)c1ccccc1)C(=O)O)OCC1c2ccccc2-c2ccccc21] |
| Fmoc-L-Glu(OtBu)-OH | Fluorenylmethyloxycarbonyl-O-t-butyl ester-L-glutamic acid |
| | 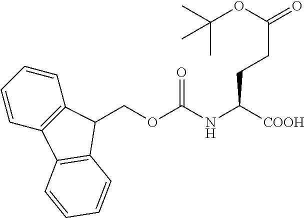 [SMILES: CC(C)(C)OC(=O)CC[C@H](NC(=O)OCC1c2ccccc2-c2ccccc21)C(=O)O] |
| Fmoc-L-Leu-OH | Fluorenylmethyloxycarbonyl-L-leucine |
| | 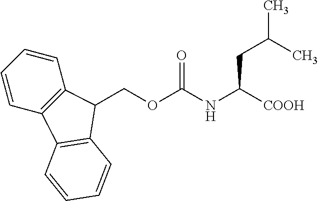 [SMILES: CC(C)C[C@H](NC(=O)OCC1c2ccccc2-c2ccccc21)C(=O)O] |
| Fmoc-L-Cth[3-Boc-L- | 2-Fluorenylmethyloxycarbonyl, 3′(t-butyloxycarbonyl-S-trityl-L- |
| Cys(Trt), 4-OAllyl]-OH | cysteine), 4′O-Allyl ester-L-cystathionine |
| tripeptide building block | 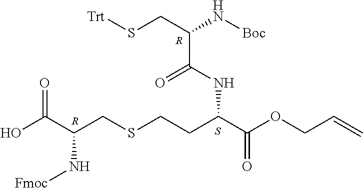 |
| Fmoc-L-Tyr(tBu)-OH | Fluorenylmethyloxycarbonyl-t-butyl-L-tyrosine |
| | 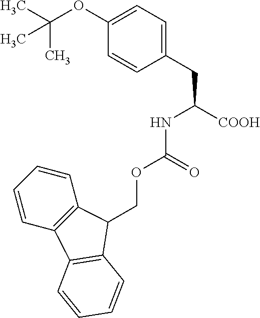 [SMILES: CC(C)(C)Oc1ccc(C[C@H](NC(=O)OCC2c3ccccc3-c3ccccc32)C(=O)O)cc1] |
| Fmoc-L-Val-OH | Fluorenylmethyloxycarbonyl-L-valine |
| | 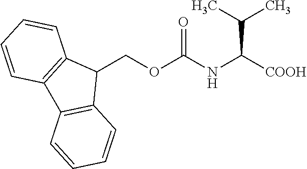 [SMILES: CC(C)[C@H](NC(=O)OCC1c2ccccc2-c2ccccc21)C(=O)O] |
| H-L-Cys(Trt)-OtBu | S-trityl-L-cysteinyl-O-t-butyl ester |
| | 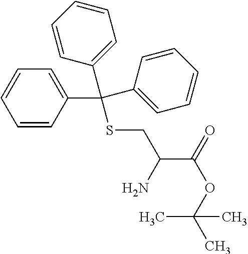 [SMILES: CC(C)(C)OC(=O)C(N)CSC(c1ccccc1)(c1ccccc1)c1ccccc1] |
| H-Gly-2-CT-Resin | H-glycine-2-chlorotrityl resin |
| | 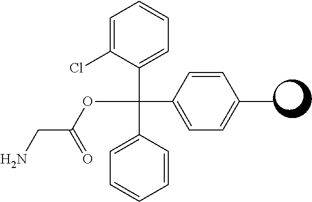 [SMILES: Cc1ccc(C(OC(=O)CN)(c2ccccc2)c2ccccc2Cl)cc1] |

TABLE 2
List of Reagents

| Abbreviation | Name |
|---|---|
| Ac₂O | Acetic anhydride |
| AcOSu | acetyl-N-hydroxysuccinimide |
| AllocCl | allyloxycarbonyl chloride |
| DIC | N,N′-diisopropylcarbodiimide |
| DIPEA | N,N-Diisopropylethylamine |
| DTT | Dithiothreitol |
| HFIP | Hexafluoro isopropanol |
| HOPO | 2-Hydroxypyridine-N-oxide |
| NaH₂CO₃ | Sodium bicarbonate |
| NH₄I | Ammonium iodide |
| NH₄OAc | Ammonium acetate |
| NH₄OH | Ammonium hydroxide |
| HO-Su | N-hydroxysuccinimide |
| Oxyma ® | Ethyl 2-cyano-2-(hydroxyimino) acetate |
| Pd(PPh₃)₄ | Tetrakis (triphenylphosphine) Palladium (0) |
| N/A | Phenylsilane |
| N/A | Piperidine |
| N/A | Pyridine |
| Si-Thiol | 1-Propanethiol-Silica |
| TBTU | 2-(1H-Benzotriazol-1-yl)-1,1,3,3-tetramethyluronium |
| | tetrafluoroborate |
| N/A | Thioanisole |
| TIS | Triisopropyl silane |

TABLE 3
List of Solvents

| Abbreviation | Name |
|---|---|
| 2-MeTHF | 2-Methyl tetrahydrofuran |
| ACN | Acetonitrile |
| AcOH | Acetic acid |
| DCM | Dichloromethane |
| DMF | N,N-Dimethylformamide |
| DMSO | Dimethyl sulfoxide |
| H₂O | Water |
| IPA | Isopropyl alcohol (isopropanol) |
| MeOH | Methanol |
| MTBE | Methyl-tert-butyl ether |
| N/A | n-Heptane |
| NMP | N-methyl-2-pyrrolidone |
| TFA | Trifluoroacetic acid |
| THF | Tetrahydrofuran |

### Example 1

### Manufacturing Process of Synthetic Peptide of SEQ ID NO: 1

### Introduction

The peptide of SEQ ID NO: 1, an N-terminally modified peptide of SEQ ID NO: 1, or a pharmaceutically acceptable salt thereof, is to be used in planned clinical studies and was manufactured in compliance with Good Manufacturing Practice (GMP) regulations. All abbreviations are listed in Table 2 (List of reagents) and Table 3 (List of solvents), respectively.

The peptide of SEQ ID NO: 1, an N-terminally modified peptide of SEQ ID NO: 1, or a pharmaceutically acceptable salt thereof, was manufactured according to the synthetic scheme described below. The route of synthesis combined the stepwise synthesis of part of the peptide chain according to well established principles of solid-phase peptide chemistry. This was followed by successive steps to incorporate the C-terminal amino acid, cyclization to form the thioether bridge, folding, primary purification, N-terminal acetylation, final purification by preparative-scale chromatography, and isolation of the drug substance as a solid by precipitation.

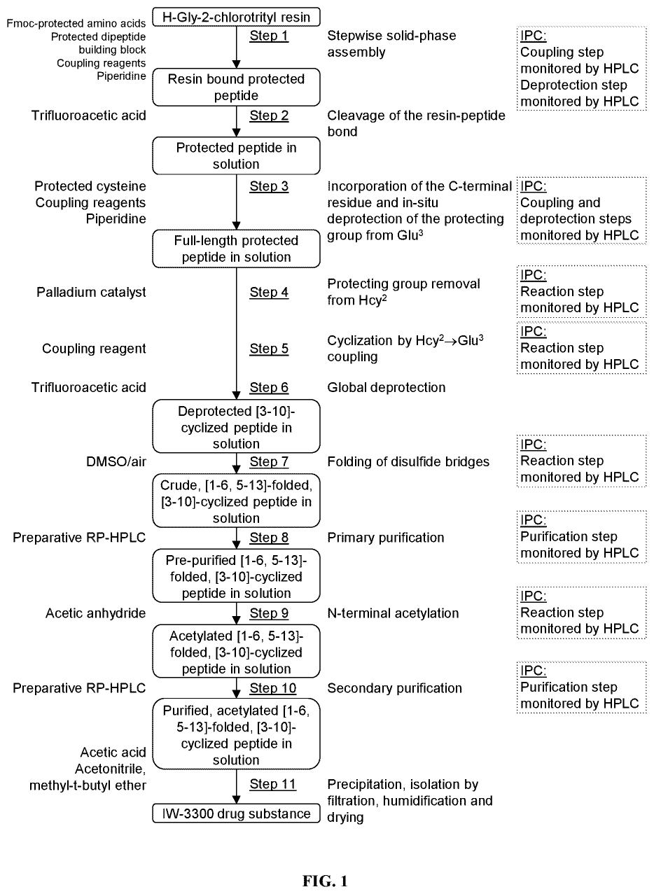

An exemplary manufacturing process for the peptide of SEQ ID NO: 1, an N-terminally modified peptide of SEQ ID NO: 1, or a pharmaceutically acceptable salt thereof, is presented in FIG. 1. All starting materials composing the peptide primary sequence are introduced under a protected form compatible with the selected chemistry. All optically active amino acid residues are used under the naturally occurring “L” form.

The folding leading to the two disulfide bridges between designated cysteine residues is induced after completion of the assembly. The thioether bond between the homocysteine (in position 2) and the cysteine (in position 10) side chains is pre-formed in a tripeptide building block. The subsequent cyclization is induced by the formation of a lactam bond as described below (step 5). After the final chromatographic purification, the drug substance is isolated by precipitation and drying.

### Peptide Synthesis

### Step 1: Stepwise Solid Phase Assembly

The primary amino acid sequence was assembled by an iterative process starting from a 2-chlorotrityl resin pre-loaded with a glycine residue (Gly₁₂ of the synthetic peptide). Nine successive cycles, detailed in Tables 4-5, were conducted as follows:
Removal of the N-terminal Fmoc protecting group from the previously introduced amino acid residue by treatment with a base (piperidine) in dimethylformamide (DMF), followed by extensive washing with DMF. This deprotection was not executed after the last incorporation step (glutamic acid). The deprotection reactions were monitored by HPLC.
Coupling of the next Fmoc-protected amino acid or building block, suitably protected on its side chain as relevant, in NMP under the action of DIC in presence of Oxyma®, except for the first reaction (coupling of tyrosine on the glycine anchored on the resins) conducted in NMP under the action of TBTU in the presence of DIPEA. The coupling reactions were monitored by HPLC.
Extensive washing with DMF.

TABLE 4
SPPS cycles and coupling conditions.

| Cycle | AA | Coupling Conditions |
|---|---|---|
| 0 | H-Gly-2CT-Resin | 4 × 5 min Swelling |
| 1 | Fmoc-L-Tyr(tBu)-OH | TBTU, Single, 3 h |
| 2 | Fmoc-L-Tripeptide | DIC, 1.2 eq, 6 h, no pyridine |
| 3 | Fmoc-L-Ala-OH | DIC, 2.0 eq, 3 h |
| 4 | Fmoc-L-Val-OH | DIC, 2.0 eq, 3 h |
| 5 | Fmoc-L-Asn(Trt)-OH | DIC, 2.0 eq, 6 h |
| 6 | Fmoc-L-Cys(Trt)-OH | DIC, 2.0 eq, 3 h, no pyridine |
| 7 | Fmoc-L-Cys(Trt)-OH | DIC, 2.0 eq, 6 h, no pyridine |
| 8 | Fmoc-L-Leu-OH | DIC, 2.0 eq, 6 h |
| 9 | Fmoc-L-Glu(OtBu)-OH | DIC, 2.0 eq, 24 h |

Final Wash and drying of resin (30° C., p > 10 mbar)

TABLE 5
Detailed description of steps in Table 4.
Cycle 1: 1^{st} Coupling - TBTU, Single, 3 h

| | |
|---|---|
| Coupling | 2.0 eq. Fmoc-Tyr(tBu)-OH, 1.95 eq. TBTU, 3.92 eq. |
| | DIPEA in NMP, 10 min pre-activation, 3 h coupling |
| Wash | 4 × DMF (6.5 mL/g (resin)) |

Cycle 2: Tripeptide Coupling

| | |
|---|---|
| Coupling | 1.134 eq. contained Fmoc-Tripeptide, 1.8 eq. DIC, |
| | 1.2 eq. Oxyma in NMP, 10 min pre-activation, 6 h |
| Wash | 4 × DMF (6.5 mL/g (resin)) |

Cycles 3-5, 8, and 9:

| | |
|---|---|
| Coupling | 2.0 eq. Fmoc-AA-OH, 3.0 eq. DIC, 2.0 eq. Oxyma in |
| | NMP, 10 min preactivation, 3.6 eq. pyridine, 3 h/ |
| | 6 h/24 h coupling |
| Wash | 4 × DMF (6.5 mL/g (resin)) |

Cycles 6 and 7:

| | |
|---|---|
| Coupling | 2.0 eq. Fmoc-AA-OH, 3.0 eq. DIC, 2.0 eq. Oxyma in |
| | NMP, 10 min preactivation, 3 h/6 h coupling |
| Wash | 4 × DMF (6.5 mL/g (resin)) |

DeFmoc

| | |
|---|---|
| Deprotection | 3 × 10 min, 20% piperidine in NMP (6.5 mL/g (resin)) |
| Wash | 5 × DMF (6.5 mL/g (resin)) |

Final Wash

| | |
|---|---|
| IPA wash | 6 × 5 min IPA (6.5 mL/g (resin)) |
| MTBE wash | 6 × 5 min MTBE (6.0 mL/g (resin)) |

### Coupling

NMP was used as the solvent for dissolving the amino acids, DeFmoc-solution, dissolve the coupling agent, the capping solution, and the pyridine-kick. The pyridine-kick is the addition of 3.6 eq. of pyridine for each coupling cycles to avoid partial cleavage of the growing peptide from the solid support. The pyridine-kick was used in all coupling reactions except when coupling cysteine residues as discussed below. DMF was only used for washing steps after couplings and Defmoc-steps. Because NMP was used rather than DMF, the coupling time needed to be extended from 90 min. to 180 min. for standard amino acids and from 180 min. to 360 min. for the tripeptide building block. Only 1.2 equivalents of the tripeptide were used instead of 2.0 equivalents for standard couplings. The conditions for the standard process are shown in Table 4. The SPPS of the protected linear peptide was carried out as described in the protocol (Table 5) at 0.75 mmol scale on Tribute automated synthesizers with preloaded H-Gly-CT resins. The pyridine-kick was used to minimize premature cleavage of the peptide from the resin because of acidic coupling conditions.

Analytical HPLC LC-MS analytics show no hint for high content of deletion sequences. The experiments showed that 1.2 equivalents of the tripeptide building block was suitable for complete coupling.

### Racemization of Cysteines (Pyridine-Kick)

SPPS using DIC/Oxyma, which leads to acidic coupling conditions can cause partial cleavage of the peptide from the resin. Therefore, a pyridine-kick was established. As indicated above, a pyridine kick is the addition of a portion of pyridine to push the reaction to completion. It is known, that racemization can occur for basic couplings of Cysteines. Therefore, the pyridine-kick was not implemented during the coupling of cysteine residues as they are especially sensitive to alkaline conditions (such as induced by pyridine) with respect to the risk of racemization. Because of that, a test peptide using methionine instead of the L-Tripeptide was synthesized once with and once without pyridine-kick for cysteine-couplings.

Using the pyridine-kick for all couplings lead to a content of D-cysteine of 1.49% and 1.35% for the peptide synthesized without pyridine-kick at cysteine-couplings. Also, there was no influence for on resin yield of the SPPS. To conclude, the influence on yield and quality of the obtained product was minimal. To reduce the risk of racemization, however, no pyridine-kick was used for cysteine- and tripeptide (chemically similar to protected cysteine) couplings.

### Loading of Resin

The influence of the resin loading was investigated while generating new material for downstream processing. In one experiment, a preloaded H-Gly-CT resin with a loading of 0.7 mmol/g was used, while in another experiment a preloaded H-Gly-CT resin with a loading of 0.9 mmol/g was used. The crude peptides show the same quality and the same yield on resin.

### SPPS Run

The build was run at 7.5 mmol scale on a preloaded H-Gly-2CT resin at 0.65 mmol/g. Each coupling step was monitored by Kaiser test. The results are given in Table 6. A mild test cleavage was performed and analyzed by UHPLC. A purity of 87.3% AN was reached. Yield on resin was 98%.

A second SPPS batch was run automated. The scale was again 7.5 mmol and the resin loading was 0.69 mmol/g. A purity of 89.4% AN was achieved with a quantitative (≥100%) yield on resin.

TABLE 6
Kaiser test results for SPPS run.

| | Total Coupl. | One letter | | Weight | A | |
|---|---|---|---|---|---|---|
| Cycle | Time | code | Fmoc-AA-OH | sample | (570 nm) | uncoupled |
| 1 | 3 h | Y | Fmoc-L-Tyr(tBu)-OH | 16, 10 mg | 0.284 | 0.8% |
| 2 | 6 h | X | Fmoc-Tripep | 16, 10 mg | 0.2135 | 0.9% |
| 3 | 3 h | A | Fmoc-L-Ala | 26, 30 mg | 0.2166 | 0.6% |
| 4 | 3 h | V | Fmoc-L-Val-OH | 30, 30 mg | 0.3482 | 0.8% |
| 5 | 6 h | N | Fmoc-L-Asn(Trt)-OH | 46, 40 mg | 0.4653 | 0.8% |
| 6 | 3 h | C | Fmoc-L-Cyc(Trt)-OH | 35, 40 mg | 0.3444 | 0.9% |
| 7 | 6 h | C | Fmoc-L-Cys(Trt)-OH | 23, 10 mg | 0.1895 | 0.9% |
| 8 | 3 h | L | Fmoc-L-Leu-OH | 27, 30 mg | 0.1331 | 0.2% |
| 9 | 3 h | E | Fmoc-L-Glu(OtBu)-OH | 11, 10 mg | 0.214 | 0.9% |

### Step 2: Cleavage of the Resin-Peptide Bond

The peptide is cleaved from the resin under a mild acidic treatment that preserves the side chain protecting groups as well as the N-alpha Boc protecting group of the Cys₁ residue. The completion of this reaction is time specific. The reaction mixture is concentrated by evaporation and the solvent is exchanged to DMF.

To avoid formation of gummy solid, sticking to the glassware, the direct coupling of the C-terminal Cysteine was tested. This omitted one isolation step resulting in a shorter cycle time. The direct coupling was tested with a mild cleavage batch, where different anti-solvents were already tested in order to precipitate the peptide. The batch was evaporated to an oil, reconstituted with DCM and again evaporated in order to remove all residual anti-solvents. After reconstitution with DCM, the peptide was used as is for Cysteine coupling. Table 7 shows the cleavage conditions.

TABLE 7
Conditions for mild resin cleavage.

| Step | Conditions |
|---|---|
| Pre-Swell | 1 × 10 min, 3.6 Veq (on resin) DCM, drain |
| Acid treatment | 2 × 20 min, 1% TFA in DCM, 4Veq (on resin) each |
| Neutralization | Add after both treatments were combined: |
| | 50% Pyridine in DCM, 160 mL Pyridine/kg(resin) |
| Resin Wash | 3 × 5 min, DCM, 1 Veq (on resin) each |
| | Add to cleavage |
| Extract | 2 × with same volume water |
| Evaporation | Evaporate to 3 Veq (on peptide), 35° C. water bath, |
| | 150 mbar |
| Solvent Swap | Add 5 Veq (on peptide) DMF |
| Evaporation | Evaporate to at least 7 Veq (on peptide), 35° C. water |
| | bath, 150 mbar |
| Next | The solution is ready for the next step |

Step 3: Incorporation of the C-Terminal Residue and In-Situ Deprotection of the Protecting Group from Glu₃

The cleaved peptide solution from step 2 was activated by DIC/HOPO, and the coupling with H-Cys(Trt)-OtBu was conducted in DMF in the presence of DIEA as base. When the reaction was complete, as monitored by PLC, the Fmoc protecting group of the N-alpha Glu₃ residue was cleaved by the addition of piperidine directly to the reaction mixture. Upon completion of the reaction as monitored by PLC, the product of the reaction was isolated by extraction, precipitation, filtration, and drying. Table 8 shows the cysteine coupling conditions.

TABLE 8
Cysteine coupling conditions.

| Step | Conditions |
|---|---|
| Dissolution | Protected peptide from cleavage is used as is in DMF |
| | Transferred if needed, 1 Volume DMF for rinse or add to reach 8 |
| | Volumes in total |
| Preactivation | HOPO (3.0 eq) and DIC (1.5 eq) are added neat, stir for 15 min |
| Coupling | H-Cys(Trt)-O-tBu (1.5 eq) with DIEA (1.4 eq) in DMF, 2Veq is |
| | added, stir over night and monitor by HPLC. [Add additional DIC |
| | (0.5 eq) kickers after 10 h intervals if needed] |
| DeFmoc | Add 10 eq of Piperidine and stir for 30 min |
| Dilute | Add same volume 2MeTHF/Hexanes (20/1) |
| Extract | 2 × with 5 Veq Water |
| Concentration | Evaporate to 4 Veq, 35° C. water bath, 150 mbar |
| Re-Evaporation | Add 2Veq 2-MeTHF and strip down to 4 Veq |
| Precipitation | Slowly add 8 Veq of Heptane (30 min) at RT |
| Ageing | Stir at RT for 10 min |
| Isolation | Filter off, wash 4 × with 0.5Veq Heptane |
| Drying | Dry in vacuum at RT |

Step 4: Protecting Group Removal from Hcy₂

The O-allyl ester protecting group of the C-alpha Hcy₂ was subsequently removed by dissolving it in DCM and cleaving the O-allyl ester with a palladium-containing catalyst (Pd(PPh₃)₄) in the presence of phenyl silane as scavenger. The progress of the reaction was monitored by HPLC. The DeAllyl conditions are as follows:
1) Dissolve Pd(PPh₃)₄ (0.05 eq) in DCM
2) Add phenylsilane (2 eq) and 13 mer to Pd(PPh₃)₄/DCM solution
3) Stir for 1 h and monitor the reaction progress by HPLC
4) The solution is ready for the next step when the de-allylation is done.
   Step 5: Cyclization by Hcy₂→Glu₃ Coupling

Upon completion of step 4, HOPO and DIC were directly added to the reaction mixture to induce the cyclization by the coupling between the carboxylic function of Hcy₂ and the amine function of Glu₃. This reaction was monitored by HPLC. Table 9 shows the cyclization conditions.

For all cyclization concentrations (5, 10, 25, or 50 mg/mL), no peaks of oligomers were visible in the chromatograms. All four experiments were run at the same scale, the crude yield for each experiment was 40±2 mg. The largest peak area was obtained for the cyclization at 50 mg/mL indicating the highest amount of peptide contained in the crude.

Cyclization was also tested at 100 mg/mL. In order to achieve this, coupling agents were directly added to the DeAllyl solution. Judging by the chromatograms, no peaks showing oligomerization are visible. Both cyclization concentrations (50 and 100 mg/mL) give very similar results. The higher concentrations did not give any benefit in terms of purity.

TABLE 9
Cyclization conditions.

| Step | Conditions |
|---|---|
| Dissolution | Protected peptide dissolved in DCM coming from |
| | DeAllyl is used |
| Preparation | HOPO (4.0 eq) and DIC (2.0 eq) are added, stir at RT |
| Monitor and Re- | Monitor reaction by HPLC, add additional DIC kicker |
| activate | (0.5 eq) if needed |
| Next | The solution is ready for the next step |

### Step 6: Global Deprotection

### Pd Removal

Once the cyclization was complete, the reaction mixture was concentrated by evaporation under vacuum (to about 50% of the original volume) and was treated by Si-thiol, a scavenger specific for the removal of the Pd-based catalyst. The complexed Pd-scavenger was filtered off and the collected filtrate was further concentrated by evaporation. Table 10 shows the Pd removal conditions.

TABLE 10
Pd Removal Conditions.

| Step | Conditions |
|---|---|
| Concentration | The DCM solution from cyclization is concentrated to |
| | ½ of its original volume |
| DePd | Add Si-Thiol (10 eq to Pd) to concentrate and stir |
| | overnight |
| Filtration and | Filter the scavenger off, wash 3 × with 1 Veq DCM |
| wash | |
| Concentration | Reduce the volume to 2 Veq based on contained |
| | peptide mass |

The still-protected, cyclized 13-mer peptide was then subjected to a complete deprotection by the addition of a mixture of DTT, water, thioanisole, TIS, and NH₄I in TFA. Upon completion of the reaction, the crude, deprotected peptide was recovered by precipitation in n-Heptane and MBTE. The solid obtained was dried under vacuum. Table 11 shows the global deprotection conditions.

TABLE 11
Conditions for Global Deprotection.

| Step | Conditions |
|---|---|
| Peptide | The peptide in DCM is used as is from the Pd removal step |
| Solution | |
| Cocktail | Make a 10 volume (to weight of 13mer) cocktail solution of |
| | TFA/DTT/water/thioanisole/TIS/NH₄I (80/2/8/6/4/0.05, v/w/v/v/v/w) |
| Cleavage | Add cocktail to the peptide solution and stir for 3 h |
| Precipitation | Add cold heptane (0° C., 3 Volumes to cleavage cocktail) to the mixture |
| | Add cold MTBE (0° C., 2 Volumes to cleavage cocktail) to the mixture |
| Ageing | Mix the solution for 0.5 h at 0° C. |
| Isolation | Filter the peptide off, wash 4 times with 0.5 Volume MTBE, dry under |
| | reduced pressure at RT |

### Step 7: Folding by Disulfide Bridges

The crude deprotected peptide from step 6 was added to ammonium bicarbonate and DMSO is added to induce the folding by the formation of the two disulfide bridges between the side chains of Cys₁ and Cys₆, and Cys₅ and Cys₁₃, respectively. The folding reaction was monitored by HPLC. Table 12 shows the conditions used for the folding step.

TABLE 12
Conditions for Peptide Folding

| Step | Conditions |
|---|---|
| | Target concentration 0.5 g/L contained peptide in DMSO/50 mM |
| | AMBIC = 1/9, free amine. |
| Peptide Slurry | Add peptide to 50 mM AMBIC buffer |
| Oxidation | Add DMSO, peptide will dissolve, let stir for 48 h |
| Acidification | The pH is set to 1-1.5 with TFA, precipitation will occur |
| Filtration | Filter slurry over Whatman GF6 filter to obtain clear solution |
| | ready for HPLC purification - filtration is very slow |

### Step 8: Primary (1^{st}) Purification

Upon completion of step 7, the reaction mixture was acidified with TFA, celite was added as a filtration aid and the resulting slurry is filtered. The clear filtrate was directly loaded on a preparative HPLC column packed with C18 (3) stationary phase. The purification was conducted by a gradient elution in 0.1% TFA in acetonitrile and 0.1% TFA in acetonitrile:water (5:95 v/v). Individual fractions were collected, and the selection was based on analytical HPLC monitoring: fractions exhibiting a purity of ≥90% (area %) are pooled.

### Step 9: N-Terminal Acetylation

The direct acetylation of the folded peptide in the folding solution was tested. After addition of 15 eq of AcOSu, only very little conversion was detected. After pH adjustment, and additional AcOSu charges, still no substantial conversion was obtained. This was repeated with a second folding solution with the same result. The addition of Ac₂O did not result in a better conversion.

To get an impression on the acetylation reaction of this compound, two experiments were run overnight, using 15 eq AcOSu and 2 eq Ac₂O, respectively. Both reactions showed promising results, the reaction with AcOSu was not complete after 15 h.

The reaction was repeated with increased equivalents of Ac₂O, 3 eq and 4 eq, respectively, and monitored after 18 h. The higher amount of acetylation agent seemed to push the reaction to completion and the reduced reaction time seemed to lower the amount of side reactions, especially for the two biggest late eluting impurities.

Accordingly, the final process was defined to run with 4.0 eq Ac₂O. Therefore, the acetylation step 9 was run from the collected pool from the primary (1^{st}) purification step 8, which was diluted in an ammonium bicarbonate solution containing DMSO and the N-alpha Cys₁ residue is then acetylated by acetic anhydride. The reaction was monitored by HPLC. Table 13 shows the conditions for the acetylation step.

TABLE 13
N-terminal acetylation conditions

Step

| | |
|---|---|
| Dilution | Main Pool from first pass is diluted with same volume 50 |
| | mM AMBIC/DMSO = 9/1 |
| Acetylation | Ac₂O (4 eq) are added to the pool at RT |
| Monitor and re- | Monitor reaction using IM3, 2 μL injection |
| acetylate | Add additional Ac₂O if reaction is incomplete |

### Step 10: Secondary Purification

The acetylation reaction mixture was diluted 1/1 in an ammonium acetate solution, the pH is adjusted to 7-8 with a 30% (v/v) aqueous solution of ammonium hydroxide and subjected to the final purification by injection on a preparative HPLC column packed with C18 (3) stationary phase. The purification was conducted by a gradient elution in acetonitrile and 25 mM aqueous ammonium acetate:acetonitrile (95:5, v/v).

Individual fractions were collected, and the selection was based on analytical HPLC monitoring: fractions exhibiting a purity of ≥95% (area %) and no single impurity>1.0% (area %) are pooled.

With the optimized acetylation conditions to reduce the starting material below 0.1%, the 2^{nd} purification provided slight polishing of the material and facilitated the ion exchange from trifluoroacetate to acetate.

### Step 11: Precipitation, Isolation by Filtration and Drying

The collected purified pool from the secondary purification step was concentrated by evaporation under vacuum to a target concentration of 50-100 mg/mL. The product was then precipitated by acidification (acetic acid), dilution with acetonitrile, and addition of MBTE. The precipitate was recovered over a 0.2 μm filter and thoroughly washed with a MBTE/acetonitrile solution. The conditions are shown in Table 14.

The solid was humidified with 30% (v/v) aqueous acetonitrile followed by water and isolated by final drying under vacuum yielding the synthetic peptide of SEQ ID NO: 1.

TABLE 14
Conditions for Step 11.

| Step | Conditions |
|---|---|
| Concentration | Evaporate at 35° C. water bath to a target concentration |
| | of 100-50 mg/mL |
| AcOH addition | Add 0.75 × Volume of AcOH to the pool |
| ACN addition | Dilute to 6% Water, assuming water content to be 100% |
| | after stripping the ACN off |
| MTBE addition | Add same volume MTBE compared to ACN |
| Ageing | Stir at RT for 30 min |
| Filtration | Filter off using Nylon membrane 0.2 μm |
| Wash | Wash using ACN/MTBE = 1/1, 2 × 1/10 of initial pool |
| | volume |
| Drying | Dry at 25° C. under reduced pressure |

### Example 2

### Preparation of Alloc-HCys(Fmoc-Ala-OH)-3-yl)-OAll

The following reaction scheme shows the preparation of the title compound:

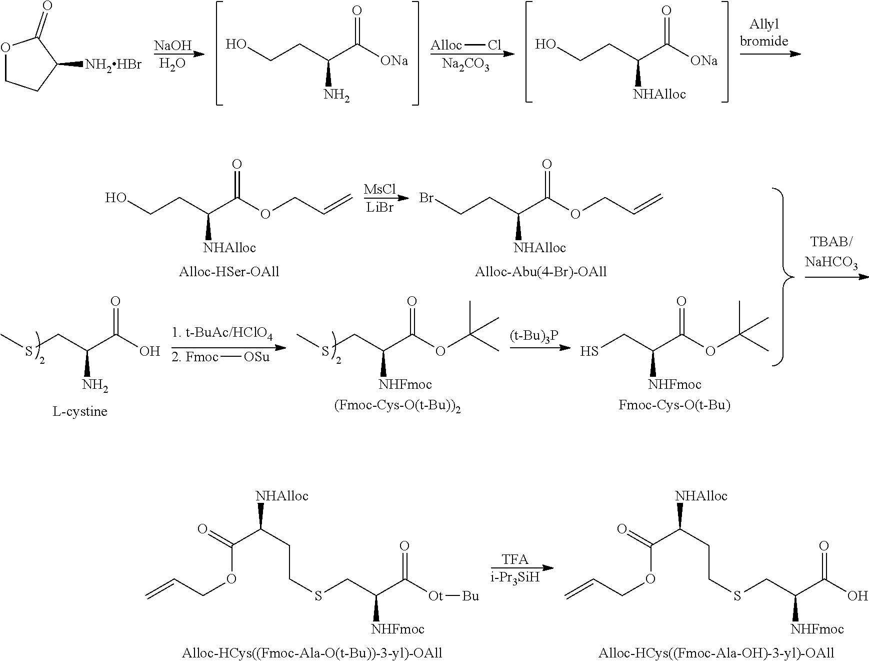

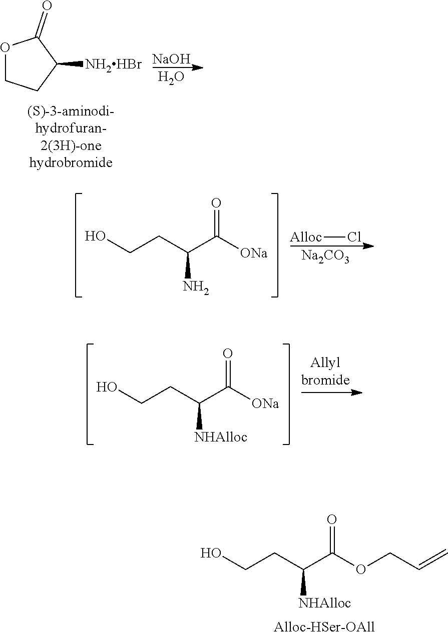

In a 5 L three-neck-flask, 500 g of (S)-3-aminodihydrofuran-2(3H)-one hydrobromide salt was dissolved in 1 L of water. A solution of 230 g NaOH in 1 L of water was added. The reaction was monitored by TLC. After the reaction was finished, the pH was adjusted to 7.5 by adding conc. HCl. 230 g of Na₂CO₃ was added, and the mixture was cooled to 0° C. 331 g of alloxycarbonyl chloride (Alloc-Cl) was added dropwise, in the meantime the temperature was kept at 0-10° C., and the pH was kept at 7.5. The mixture was stirred overnight. The solvent was then removed, and then 2500 mL of DMF was added. After 230 g of NaHCO₃ was added, 665 g of allyl bromide was added. The mixture was stirred overnight, before 7000 mL of water was added to quench the reaction. The mixture was extracted with MTBE (5 L*3), and the combined organic phase was washed with 800 mL of NaHCO₃, 800 mL of KHSO₄ and 800 mL of brine in sequence. After the solvent MTBE was removed, the residue was purified by column chromatography (SiO₂) to give 273.9 g of oily product. ¹H NMR (400 MHz, Chloroform-d) δ 6.02-5.65 (m, 3H), 5.39-5.07 (m, 4H), 4.58-4.17 (m, 4H), 3.80-3.52 (m, 2H), 3.08 (s, 1H), 2.77-2.17 (m, 1H), 2.17-2.01 (m, 1H), 1.88-1.64 (m, 1H). ESI-MS Calculated for C11H17NO5 [M+H]⁺: 244.12; Found: 244.08.

### Synthesis of Alloc-Abu(4-Br)-OAll

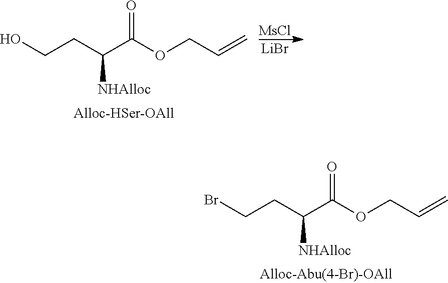

In a 10 L three-neck-flask, 378 g of Alloc-HSer-OAll and 216 g of Et₃N were dissolved in 3400 mL of DCM. 215 g of mesyl chloride (MsCl) was then added dropwise, in the meantime the temperature was kept at 0-10° C. After the reaction finished, 3400 mL of acetone was added, followed by portionwise addition of 1356 g of LiBr. The reaction was kept at 25-30° C. overnight. 200 g of LiBr and 520 mL of acetone were added to drive the reaction complete. Then the solvent was removed, and the residue was combined with another batch starting from 153 g of Alloc-HSer-OAll. 572 g of Alloc-Abu(4-Br)-OAll was obtained after column chromatography (SiO₂). ¹H NMR (400 MHz, Chloroform-d) δ 5.99-5.75 (m, 2H), 5.52 (d, J=8.2 Hz, 1H), 5.40-5.11 (m, 4H), 4.63 (d, J=5.8 Hz, 1H), 4.55 (d, J=5.4 Hz, 2H), 4.48 (td, J=8.4, 5.1 Hz, 1H), 3.42 (t, J=7.0 Hz, 2H), 2.50-2.34 (m, 1H), 2.33-2.13 (m, 1H). ESI-MS Calculated for C11H16BrNO4 [M+H]⁺: 306.03; Found: 306.11.

### Synthesis of (Fmoc-Cys-O(t-Bu))₂

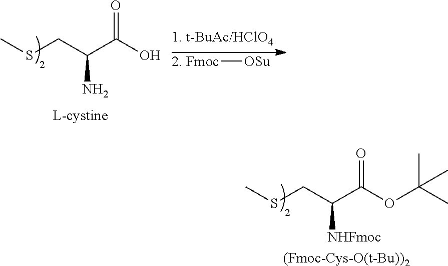

210 g of L-Cystine and 4 kg of t-butyl acetate were mixed in a 5 L three-neck-flask. 301.2 g of HClO₄ was added dropwise. The mixture was stirred overnight. 2.3 kg of K₂CO₃ and 4.0 kg of water were added. The mixture was stirred overnight, and then filtered through 20 g of celite. After separated, the organic layer was washed with 1 L of brine, and then concentrated to afford 258.8 g of oily residue. After the residue was dissolved in 2400 L of THF, 430 g of Fmoc-OSu was added. Then, 126 g of N-methylmorphine was added dropwise. The reaction mixture was stirred for 2 hours. The solvent of THE was removed, and 1500 mL of DCM was added to dissolve the residue. The organic solution was washed with 10% citric acid solution, saturated NaHCO₃ and brine in sequence. After the solvent was removed, 500 mL of EtOAc was added. The mixture was slurred for 2 hours, and then filtered. The filter cake was dried to give 453.3 g of (Fmoc-Cys-O(t-Bu))₂. The yield was 65%. ¹H NMR (400 MHz, Chloroform-d) δ 7.75 (d, J=7.5 Hz, 2H), 7.60 (d, J=7.5 Hz, 2H), 7.39 (t, J=7.3 Hz, 2H), 7.29 (t, J=7.2 Hz, 2H), 5.79 (d, J=7.8 Hz, 1H), 4.74-4.53 (m, 1H), 4.50-4.29 (m, 2H), 4.29-4.17 (m, 1H), 3.37-3.07 (m, 2H), 1.50 (s, 9H).

### Synthesis of Fmoc-Cys-O(t-Bu)

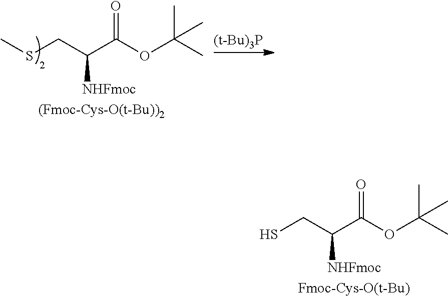

To a solution of 367 g of (Fmoc-Cys-O(t-Bu))₂ in 4300 mL of THF, 170 mL of (t-Bu)₃P was added. After the mixture was stirred for 2 hours, 550 mL of water was added to quench the reaction. The mixture was stirred overnight. The solvent of THE was removed, and 1500 mL of EtOAc was added. The mixture was stirred for 0.5 hour, and the organic was washed with 450 mL of 10% citric acid and 450 mL of brine in sequence. The organic solution was used in the next step directly.

### Synthesis of Alloc-HCys((Fmoc-Ala-O(t-Bu))-3-yl)-OAll

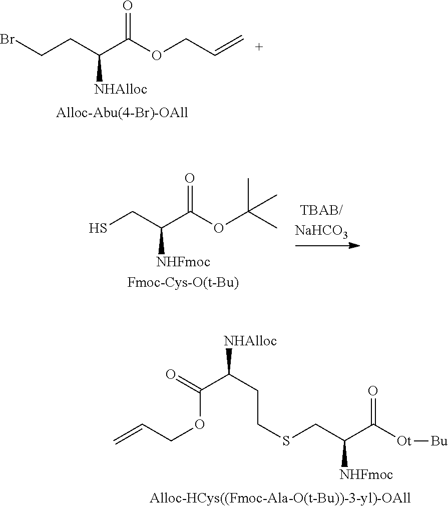

In a 10 L 4-neck-flask, 1080 g of tetrabutylammonium bromide was dissolved in 3350 mL of saturated NaHCO₃. 500 mL solution of 257 g Alloc-Abu(4-Br)-OAll in EtOAc and 2.8 L of Fmoc-Cys-O(t-Bu) solution, obtained from last step, were added. The mixture was stirred overnight. After washed with 1 L of brine, the organic solution was concentrated. 418 g of oily residue was obtained through a column chromatography (SiO₂). The yield was 80%. ¹H NMR (400 MHz, Chloroform-d) δ 7.76 (d, J=7.5 Hz, 2H), 7.61 (d, J=7.5 Hz, 2H), 7.40 (t, J=7.4 Hz, 2H), 7.31 (t, J=7.4 Hz, 2H), 5.97-5.81 (m, 2H), 5.70 (d, J=7.8 Hz, 1H), 5.43 (d, J=8.3 Hz, 1H), 5.37-5.12 (m, 4H), 4.63 (d, J=5.8 Hz, 1H), 4.61-4.43 (m, 4H), 4.39 (d, J=7.2 Hz, 2H), 4.23 (t, J=7.1 Hz, 1H), 4.12 (q, J=7.1 Hz, 1H), 3.08-2.85 (m, 2H), 2.72-2.47 (m, 2H), 2.22-2.07 (m, 1H), 2.02-1.86 (m, 1H), 1.49 (s, 9H). ESI-MS Calculated for C33H40N2O8S [M+Na]⁺: 647.24; Found: 647.24.

### Synthesis of Alloc-HCys((Fmoc-Ala-OH)-3-yl)-OAll

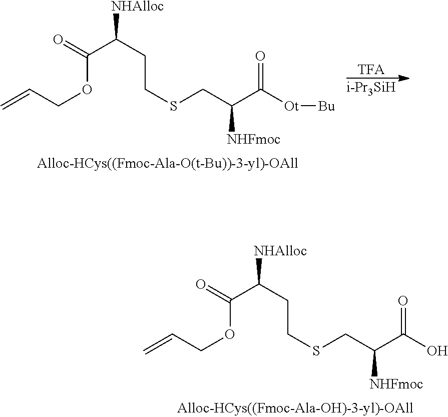

To a 5 L flask, 308 g of Alloc-HCys((Fmoc-Ala-O(t-Bu)-3-yl)-OAll, 2430 mL of TFA and 53 g of i-Pr₃SiH were added. After the solution was stirred at 20-25° C. overnight, the solvent was removed. 520 mL of DCM was added, and the solution was concentrated to remove the remaining TFA. 520 mL of DCM was added, and the solution was concentrated to remove remaining TFA. 1 L of MTBE was added to dissolve the residue. The organic solution was neutralized with 4 L of saturated NaHCO₃ aqueous solution. After the mixture was filtered, the solid was dissolved in 3 L of DCM. The organic solution was washed with acidified with 1 L of 20% citric acid solution. The resultant organic solution was washed with 900 mL of 10% citric acid solution and 500 mL of brine in sequence. The organic layer was dried over 150 g of MgSO₄, then the mixture was filtered. The solvent was removed, and the residue was stirred to solidify to give 237 g of solid product. The yield was 84.4%. ¹H NMR (400 MHz, Chloroform-d) δ 7.75 (d, J=7.5 Hz, 2H), 7.60 (d, J=7.5 Hz, 2H), 7.39 (t, J=7.5 Hz, 2H), 7.30 (t, J=7.4 Hz, 2H), 6.43 (s, 3H), 5.99-5.79 (m, 3H), 5.55 (d, J=8.3 Hz, 1H), 5.39-5.13 (m, 4H), 4.76-4.28 (m, 8H), 4.23 (dd, J=7.0 Hz, 1H), 3.22-2.76 (m, 2H), 2.74-2.48 (m, 2H), 2.14 (s, 1H), 1.97 (s, 1H). ESI-MS Calculated for C29H32N2O8S [M+H]⁺: 569.20; Found: 569.12.

### Example 3

### Preparation of Fmoc-L-Cth[3-Boc-L-Cys(Trt), 4-OAllyl]-OH Building Block

To arrive at the Fmoc-L-Cth[3-Boc-L-Cys(Trt), 4-OAllyl]-OH building block, three building blocks are synthesized separately (Parts A-C) and combined (Part D) to synthesize the title compound, as shown in the schemes below:

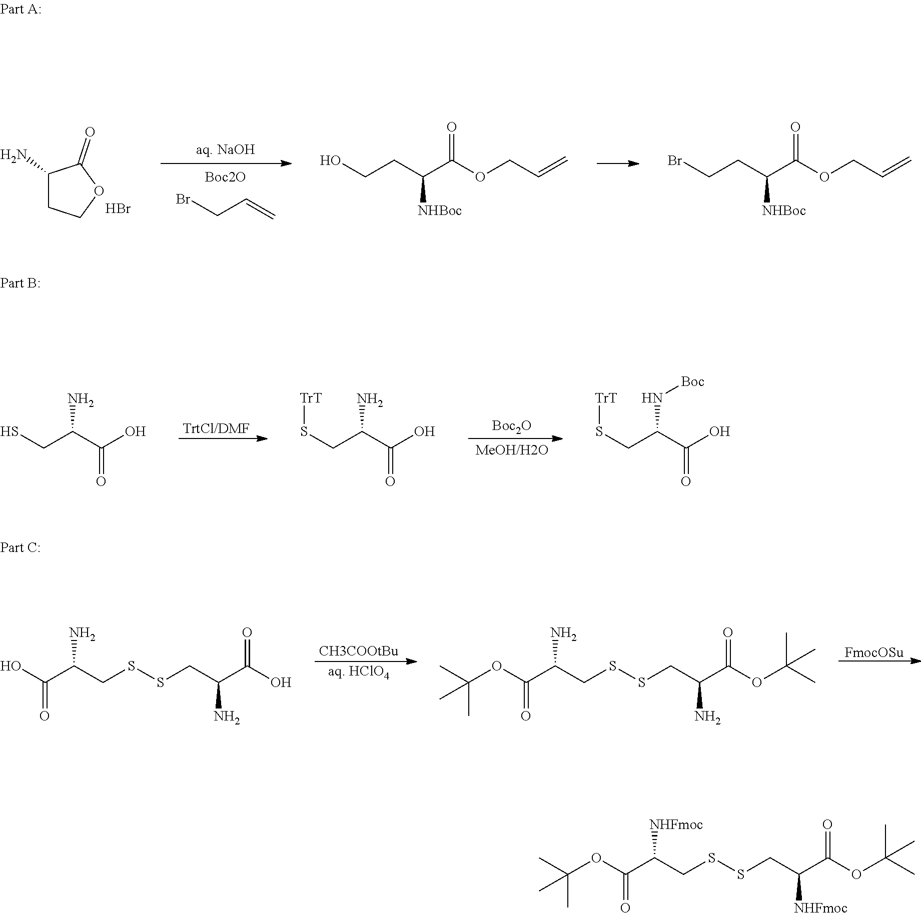

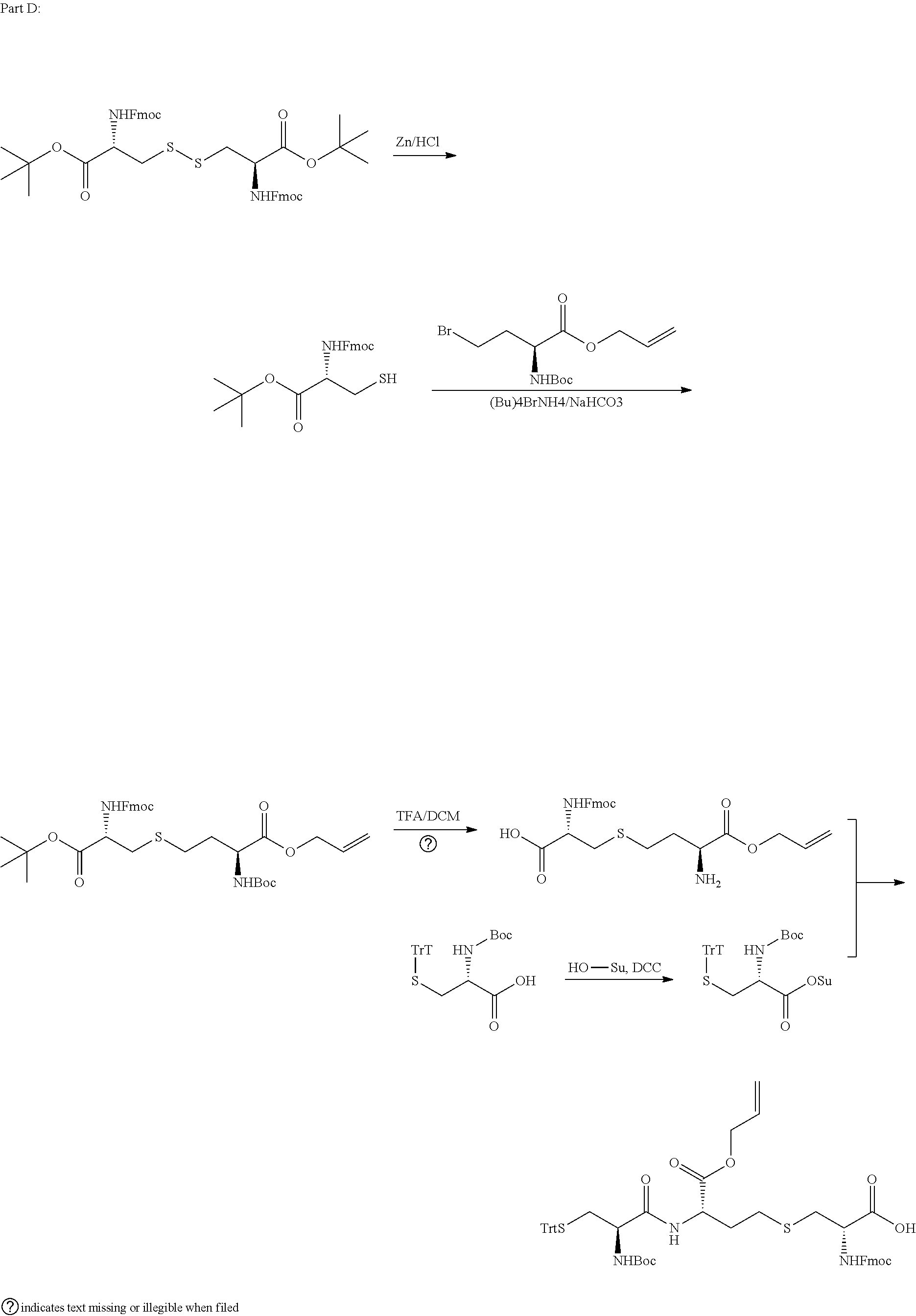

### OTHER EMBODIMENTS

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

All patents, patent applications, publications, product descriptions, and protocols are cited throughout this application, the disclosures of which are incorporated herein by reference in their entireties for all purposes.

1. A method of producing a synthetic peptide, or a pharmaceutically acceptable salt thereof, the method comprising:
(i) chemically synthesizing a linear peptide comprising a plurality of amino acids and at least one polyamino acid synthon; wherein the linear peptide has a C-terminus bound to a solid phase support;
wherein one or more amino acids of the plurality of amino acids, the at least one polyamino acid synthon, or a combination thereof, comprises a protecting group; and
wherein the at least one polyamino acid synthon comprises at least one amine group having a different protecting group from the N-terminus of the linear peptide;
(ii) cleaving the linear peptide from the solid phase support to generate a protected peptide;
(iii) coupling an amino acid to the C-terminus of the protected peptide, wherein the amino acid has an unprotected amine group, a protected carboxylic acid group, and an optionally protect amino acid side-chain;
(iv) removing one amine protecting group and one carboxylic acid protecting group from the protected peptide to form a partially unprotected peptide having an unprotected amine and an unprotected carboxylic acid group;
(v) coupling the unprotected amine and the unprotected carboxylic acid group to form a cyclized peptide;
(vi) globally deprotecting the cyclized peptide to obtain a globally deprotected peptide;
(vii) folding the globally deprotected peptide to form one or more additional crosslinks to obtain the synthetic peptide;
(viii) optionally, modifying the N-terminus of the synthetic peptide with one or more chemical moieties; and
(ix) purifying the synthetic peptide;
wherein the synthetic peptide comprises the amino acid sequence:
Cys₁ Cth₂ Glu₃ Leu₄ Cys₅ Cys₆ Asn₇ Val₈ Ala₉ Cys₁₀ Tyr₁₁ Gly₁₂ Cys₁₃ (SEQ ID NO: 1); and
wherein the synthetic peptide contains a covalent bond between the following amino acid residues of the synthetic peptide:
a) Cys₁ and Cys₆,
b) Cth₂ and Cys₁₀, and
c) Cys₅ and Cys₁₃.

2. The method of claim 1, further comprising: precipitating the synthetic peptide from solution via acidification, followed by dilution with an organic solvent mixture.

3. The method of claim 1, wherein the solid phase support is selected from the group consisting of Wang resins, Trityl resins, and Rink resins.

4. The method of claim 1, wherein the solid phase support has a loading of about 0.10 mmol/g, about 0.20 mmol/g, about 0.30 mmol/g, about 0.40 mmol/g, about 0.50 mmol/g, about 0.60 mmol/g, about 0.70 mmol/g, about 0.80 mmol/g, about 0.90 mmol/g, or about 1.00 mmol/g.

5. The method of claim 4, wherein the polyamino acid synthon is a compound represented by the following formula: 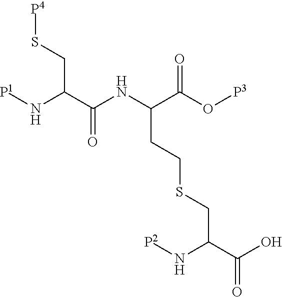
wherein:
P¹ and P² are each amine protecting group, wherein P¹ and P² are not the same;
P³ is a carboxylic acid protecting group; and
P⁴ is a thiol protecting group.

6. The method of claim 5, wherein the protecting groups are selected from fluorenylmethyloxycarbonyl (Fmoc), tert-butyloxycarbonyl (Boc), carboxybenzyl (Cbz), trityl, methyl, ethyl, tert-Butyl, allyl, 2,4-dimethoxybenzyl (Dmb), 9-fluorenylmethyl (Fm), benzyl (Bn), tert-butyldimethylsilyl, allyloxycarbonyl (alloc), tert-butyloxycarbonyl, acetamidomethyl (Acm), 3-nitro-2-pyridine sulfenyl (NPYS), or 2-pyridine-sulfenyl (Pyr).

7. The method of claim 6, wherein P¹ is tert-butyloxycabonyl (Boc); P² is 9-fluorenylmethoxycarbonyl (Fmoc); P³ is allyl; and P⁴ is trityl.

8-9. (canceled)

10. The method of claim 7, wherein the subunits of the at least one polyamino acid synthon have a D-configuration, an L-configuration, or both a D-configuration and an L-configuration.

11-12. (canceled)

13. The method of claim 10, wherein the one carboxylic acid protecting group of step (iv) is removed from the at least one polyamino acid synthon.

14. The method of claim 10, wherein the unprotected carboxylic acid group of step (v) is from the at least one polyamino acid synthon.

15. The method of claim 14, wherein the linear peptide of step (i) is synthesized by: (1) coupling at least one amino acid from the plurality of amino acids with the at least one polyamino acid synthon, or (2) by coupling at least two amino acids from the plurality of amino acids, or (3) a combination thereof; wherein the coupling occurs via a carbodiimide-mediated reaction, or a reaction mediated by a non-carbodiimide coupling agent wherein the non-carbodiimide is selected from: 1-[Bis(dimethylamino)methylene]-1H-1,2,3-triazolo[4,5-b]pyridinium 3-oxide hexafluorophosphate (HATU), (2-(1H-benzotriazol-1-yl)-1,1,3,3-tetramethyluronium hexafluorophosphate (HBTU), 1H-Benzotriazolium 1-[bis(dimethyl-amino)methylene]-5-chloro-hexafluorophosphate (1-),3-oxide (HCTU), O-(Benzotriazol-1-yl)-N,N,N′,N′-tetramethyluronium tetrafluoroborate (TBTU), 1-[(1-(cyano-2-ethoxy-2-oxoethylideneaminooxy)-dimethylamino-morpholinomethylene)]methanaminium hexafluorophosphate (COMU), 1-Cyano-2-ethoxy-2-oxoethylideneaminooxy-tris-pyrrolidino-phosphonium hexafluorophosphate (PyOxim), benzotriazol-1-yloxytripyrrolidinophosphonium hexafluorophosphate (PyBOP), 7-Azabenzotriazol-1-yloxy)tripyrrolidinophosphonium hexafluorophosphate (PyAOP), or propanephosphonic acid anhydride (T3P).

16. (canceled)

17. The method of claim 15, wherein the carbodiimide is selected from the group: diisopropylcarboxiimide (DIC), dicyclohexylcarbodiimide (DCC), or 1-Ethyl-3-(3-dimethylaminopropyl)carbodiimide (EDC).

18. (canceled)

19. The method of claim 17, wherein the carbodiimide-mediated reaction further comprises an amino acid racemization suppressing agent selected from: 2-hydroxypyridine-N-oxide (HOPO), 1-hydroxybenzotriazole (HOBt), 1-hydroxy-7-azo-benzotriazole (HOAt), or 2-cyano-2-(hydroxyimino)acetate.

20-21. (canceled)

22. The method of claim 19, wherein the carbodiimide-mediated reaction comprises: using a solvent selected from: N-methylpyrrolidinone (NMP), dichloromethane (DCM), chloroform, or dimethylformamide (DMF).

23-28. (canceled)

29. The method of claim 22, wherein the globally deprotecting step (vi) comprises adding ammonium iodide (NH₄I) and thioanisole.

30. (canceled)

31. The method of claim 130, wherein the modification of the N-terminus of the synthetic peptide is acetylation.

32. (canceled)

33. The method of claim 1, wherein the covalent bond between Cys₁ and Cys₆ and Cys₅ and Cys₁₃ is a disulfide bond; and wherein the covalent bond between Cth₂ and Cys₁₀ is a thioether bond.

34-35. (canceled)

36. The method of claim 2, wherein the organic solvent mixture comprises at least one of acetonitrile or methyl tert-butyl ether (MTBE).

37. A method of preparing a synthetic peptide of Formula I: 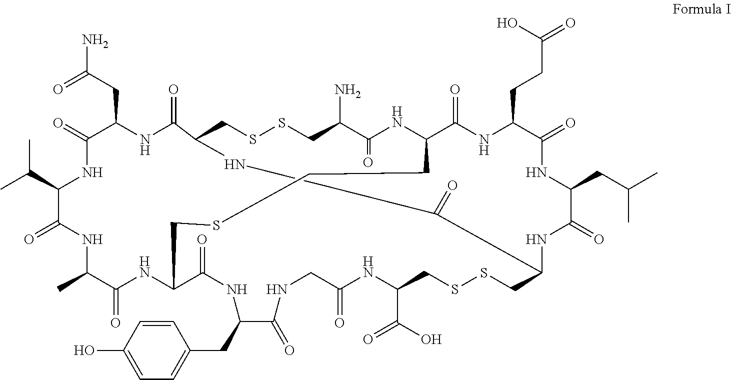 [SMILES: CC(C)C[C@@H]1NC(=O)[C@H](CCC(=O)O)NC(=O)[C@H]2CCSC[C@H]3NC(=O)[C@@H](C)NC(=O)[C@@H](C(C)C)NC(=O)[C@@H](CC(N)=O)NC(=O)[C@H](CSSC[C@@H](N)C(=O)N2)NC(=O)[C@H](CSSC[C@@H](C(=O)O)NC(=O)CNC(=O)[C@@H](Cc2ccc(O)cc2)NC3=O)NC1=O]
comprising:
(i) coupling a Tyr-Gly peptide comprising a protected Tyr amino acid to a polyamino acid synthon of Formula II: 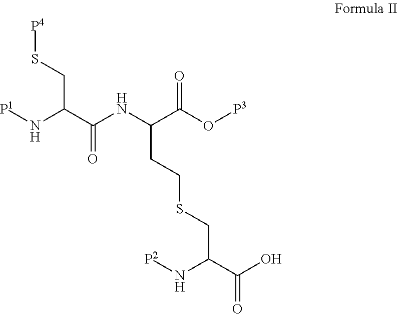
wherein the C-terminus of the Tyr-Gly peptide is bound to a resin;
wherein P¹, P², P³, and P⁴ are each protecting groups,
wherein:
P¹ and P² are each an amine protecting group, and wherein P¹ and P² are not the same;
P³ is a carboxylic acid protecting group; and
P⁴ is a thiol protecting group; and
wherein coupling the Tyr-Gly peptide to the polyamino acid synthon of Formula II results in a resin bound peptide of Formula III: 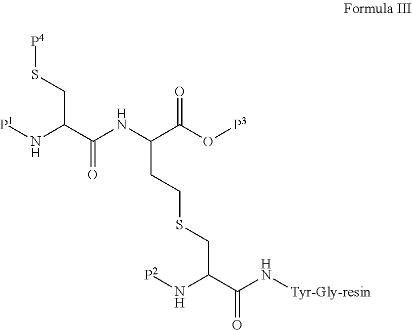
(ii) removing the P² protecting group of Formula III to obtain a resin bound peptide of Formula IV having a first free amine group: 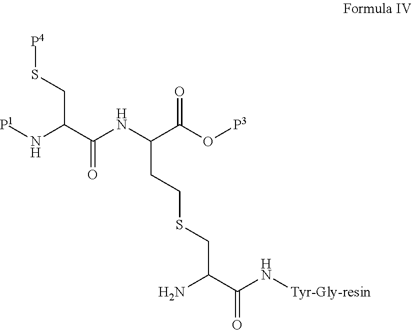
(iii) coupling a P²-alanine to the resin bound peptide of Formula IV at the first free amine group to form a resin bound peptide of Formula V: 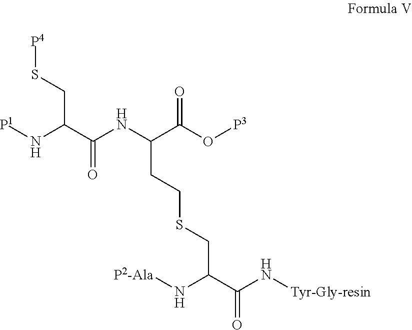
(iv) removing the P² protecting group of Formula V to obtain a second free amine group, followed by coupling the second free amine group to a P²-amino acid;
wherein the amino acid of the P²-amino acid is valine; and
wherein the side chain of the amino acid is optionally protected;
(v) repeating step (iv) for five more times, wherein the amino acid of the P²-amino acid used for each of the five more times are Asn, Cys, Cys, Leu, and Glu, respectively; to form a resin bound peptide of Formula VI: 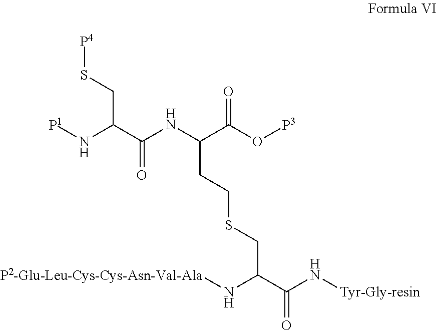
wherein at least one amino acid side chain is protected;
(vi) cleaving the resin bound peptide of Formula VI from the resin to form a linear peptide having a C-terminal carboxylic acid group;
(vii) coupling the C-terminal carboxylic acid group of the linear peptide to an amine group of a cysteine,
wherein the cysteine comprises a carboxylic acid protecting group, and
wherein a side chain of the cysteine is optionally protected,
to obtain a protected peptide of Formula VII: 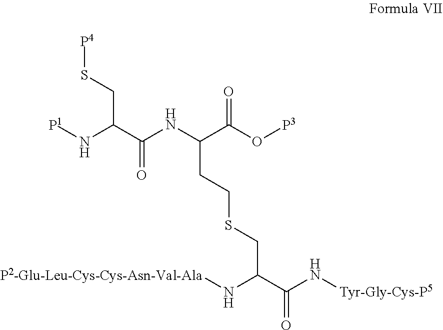
wherein P⁵ is a carboxylic acid protecting group that is different from the protecting group;
(viii) removing the P² protecting group of Formula VII to obtain a third free amine group, and the P³ protecting group of Formula VII to obtain a free carboxylic acid group;
(ix) coupling the third free amine group and the free carboxylic acid group, to obtain a cyclized peptide of Formula VIII: 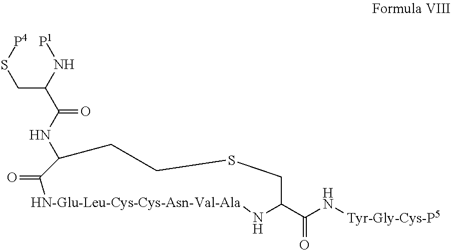
(x) globally deprotecting the cyclized peptide to obtain a globally deprotected peptide; and
(xi) folding the globally deprotected peptide by forming two disulfide bonds to obtain the synthetic peptide of Formula I.

38. The method of claim 37, further comprising acetylating a free amine group in Formula I to obtain a synthetic peptide of Formula IX:

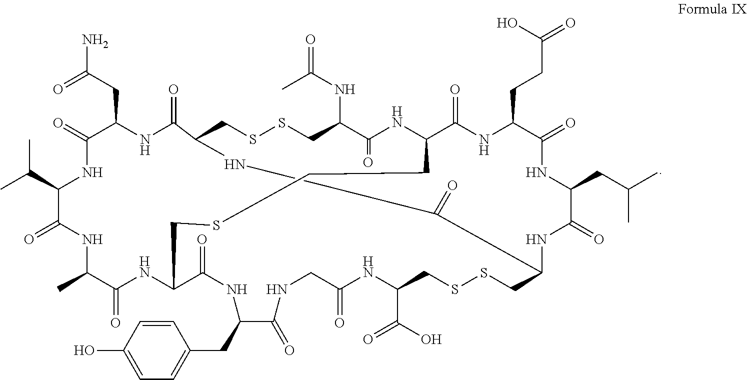 [SMILES: CC(=O)N[C@@H]1CSSC[C@@H]2NC(=O)[C@@H]3CSSC[C@@H](C(=O)O)NC(=O)CNC(=O)[C@@H](Cc4ccc(O)cc4)NC(=O)[C@@H](CSCC[C@@H](NC1=O)C(=O)N[C@@H](CCC(=O)O)C(=O)N[C@@H](CC(C)C)C(=O)N3)NC(=O)[C@@H](C)NC(=O)[C@@H](C(C)C)NC(=O)[C@@H](CC(N)=O)NC2=O]

39. The method of claim 37 or 38, wherein the Glu, Cys, Cys, Asn, Gly, and Cys residues of Formula VII have side chain protecting groups.

40-47. (canceled)

48. A compound, or a pharmaceutically acceptable salt thereof, represented by the following structural formula: 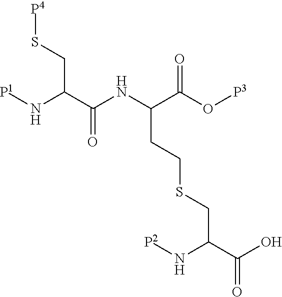
wherein:
P¹ and P² are each individually a hydrogen, or an amine protecting group;
wherein if both P¹ and P² are amine protecting groups, then the amine protecting groups are not the same;
P³ is a hydrogen or a carboxylic acid protecting group; and
P⁴ is a hydrogen or a thiol protecting group.

49. The method of claim 37, or the compound of claim 48, or the pharmaceutically acceptable salt thereof, wherein P¹ or P² are each individually an acetyl, fluorenylmethoxycarbonyl (Fmoc), tert-butyloxycarbonyl (Boc), carboxybenzyl (Cbz), or allyloxycarbonyl (Alloc); P³ is methyl, ethyl, tert-Butyl, allyl, trityl, 2,4-dimethoxybenzyl (Dmb), 9-fluorenylmethyl (Fm), or benzyl (Bn); and P⁴ is acetamidomethyl (Acm), tert-butyl (t-Bu), 3-nitro-2-pyridine sulfenyl (NPYS), 2-pyridine-sulfenyl (Pys), or trityl (Trt).

50. The method of claim 37, or the compound of claim 49, or the pharmaceutically acceptable salt thereof, wherein P¹ is an acetyl or tert-butyloxycarbonyl (Boc); P² is a fluorenylmethoxycarbonyl (Fmoc); P³ is an allyl protecting group; and P⁴ is a trityl protecting group or a tert-butyl protecting group.

51-56. (canceled)

57. The compound of claim 48, wherein at least one of P¹, P², P³, or P⁴ is a hydrogen.

58. (canceled)


SEQUENCE LISTING (3 sequences)

SEQ ID NO: 1 - protein, 13 aa, synthetic construct
  DISULFID 1..6: note = Intrachain disulfide bond
  CROSSLNK 2..10: note = Thioether bond
  VARIANT 2: note = X is homocysteine ("Hcy") or cystathionine ("Cth") that forms a cyclic thioether bond with a non-consecutive amino acid
  DISULFID 5..13: note = Intrachain disulfide bond
  SITE 1: note = Optional N-terminus acetylation
CXELCCNVACYGC

SEQ ID NO: 2 - protein, 10 aa, synthetic construct
  SITE 8: note = Residue at position 8 is linked to dipeptide comprising an N-terminus tert-butyloxycarbonyl (Boc), and C-terminus allyl ester, and creates a cystathionine thioether side chain bridge linked to Cth
  SITE 1: note = N-terminus fluorenylmethyloxycarbonyl (Fmoc)
ELCCNVACYG

SEQ ID NO: 3 - protein, 11 aa, unidentified
  SITE 8: note = Residue at position 8 is linked to dipeptide comprising an N-terminus tert-butyloxycarbonyl (Boc), and C-terminus allyl ester, and creates a cystathionine thioether side chain bridge
  SITE 11: note = C-terminus O-t-butyl-ester (OtBu)
  SITE 1: note = N-terminus fluorenylmethyloxycarbonyl (Fmoc)
ELCCNVACYGC